(19) United States
(12) Patent Application Publication
(10) Pub. No.: US 2026/0098279 A1
(43) Pub. Date: Apr. 9, 2026

(54) RECONSTITUTION OF LARGE GENES VIA CRE-LOX DNA RECOMBINATION IN ADENO-ASSOCIATED VIRUS VECTORS

(71) Applicant: University of Iowa Research Foundation, Iowa City, IA (US)

(72) Inventor: Seongjin Seo, Coralville, IA (US)

(21) Appl. No.: 19/353,409

(22) Filed: Oct. 8, 2025

Related U.S. Application Data
(60) Provisional application No. 63/704,988, filed on Oct. 8, 2024.

(51) Int. Cl. C12N 15/86; A61K 48/00; A61P 27/02; C12N 9/12

(52) U.S. Cl. CPC C12N 15/86; A61K 48/0016; A61P 27/02; C12N 9/1241; C12N 2750/14143; C12N 2830/34; C12N 2830/50

(57) ABSTRACT

The described technology pertains to gene therapy methodologies, specifically techniques and systems for delivering therapeutic genes of substantial size that exceed the packaging capacity of adeno-associated virus (AAV) vectors. The disclosed approach employs up to four AAV vectors and the CRE-lox DNA recombination system, utilizing novel lox site embodiments that allow sequence-specific and near-unidirectional recombination. This method supports efficient reconstitution of therapeutic genes up to 16 kb in a predetermined arrangement. Applications include the delivery of genes such as IFT140, PCDH15, CEP290, and CDH23 for addressing genetic disorders, including retinal degeneration. The described technology demonstrates successful production of full-length proteins in mammalian cells and mouse retinas, with therapeutic efficacy observed in an IFT140-associated retinitis pigmentosa mouse model. The CRE-lox approach offers a flexible platform for addressing AAV's packaging constraints, enabling effective gene therapy for large genes.

### PRIORITY

This application claims the benefit of U.S. Provisional Application No. 63/704,988, filed Oct. 8, 2024, the content of which is herein incorporated by reference in its entirety.

### GOVERNMENT GRANT SUPPORT

This invention was made with government support under R01-EY034176 awarded by the National Institutes of Health. The government has certain rights in the invention.

### FIELD OF THE INVENTION

The present disclosure pertains to gene therapy technologies, specifically to methods and systems for delivering large therapeutic genes using adeno-associated virus (AAV) vectors and CRE-lox recombination for the treatment of genetic disorders, including retinal degeneration.

### INCORPORATION BY REFERENCE OF SEQUENCE LISTING

This application contains a Sequence Listing which has been submitted electronically in ST26 format and hereby incorporated by reference in its entirety. Said ST26 file, created on Oct. 8, 2025, is named 875253US1.xml and is 27,032 bytes in size.

### BACKGROUND

Adeno-associated virus (AAV) is one of the most broadly used viral vectors for gene therapy. With diverse natural and engineered serotypes, AAVs can transduce a wide range of cell types, while eliciting minimal immune responses (1-4). Furthermore, AAV-mediated gene delivery appears to result in long-term transgene expression, which is an important qualification for a cure for a genetic disease. The safety, broad tropism, and long-term transgene expression have made AAV the preferred vector for gene therapy in numerous clinical trials.

However, AAV has a major disadvantage as a gene transfer vector. AAVs can accommodate up to 5 kb of viral genomes (5), and this packaging capacity is insufficient to carry full-length coding sequences of many disease-causing genes. For instance, the full-length coding sequence of ABCA4, which is the most frequently mutated gene among all Mendelian retinal degeneration genes (6), spans 6,822 bp. A ciliopathy gene CEP290 is associated with multiple genetic diseases ranging from isolated early-onset retinal degeneration to oculorenal dysplasia and lethal Meckel-Gruber syndrome (7-11). Mutations in CEP290 are one of the most frequent causes of Leber congenital amaurosis (LCA), severe vision loss at birth or in infancy, and its coding sequence is 7,440 bp-long (6, 8, 12). The coding sequence of CDH23, mutations of which cause Usher syndrome affecting both vision and hearing, spans 10,065 bp (13, 14). Since viral packaging signals (i.e., inverted terminal repeats (ITRs)) and other essential regulatory elements (e.g., a promoter and a transcription termination signal) must be included in viral vectors, the practical limit of a therapeutic gene that may be delivered via AAVs is ˜4 kb. This limited packaging capacity of AAV precludes its use as a gene transfer vector when large gene delivery is needed.

Various strategies have been developed to deliver genes larger than the packaging capacity of AAV vectors (15, 16). In these strategies, cargo genes are split and packaged in two (or three) AAV vectors for delivery to target cells. The split genes can be reconstituted at the DNA, mRNA, or protein level. The DNA-level reconstitution approach relies on occasional DNA recombination events that occur between AAV genomes after internalization. This strategy includes dual (or triple) AAV trans-splicing and hybrid methods (17-20). A new method to combine two AAV vector genomes using the CRE-lox DNA recombination system has been recently reported (21). In these approaches, the reconstituted therapeutic cassette produces mRNAs encoding full-length proteins. Additionally, REVERT (reconstitution via mRNA trans-splicing) has been reported to facilitate the reconstitution of large genes at the mRNA level (22). The protein-level reconstitution approach employs split inteins and protein trans-splicing (23-25). Protein splicing is an auto-catalytic process where an intervening protein segment (intein) excises itself from a precursor protein and attaches its two flanking regions (exteins) through a peptide bond. Some inteins are naturally split, originating from two separate genes (24, 25), and the protein trans-splicing approach utilizes these naturally split inteins. In this approach, the N- and C-terminal halves of a protein are separately delivered and produced from corresponding AAV vectors, and reconstitution is achieved via protein trans-splicing mediated by split inteins.

Although these approaches have produced some outcomes in animal models (18, 20, 22, 26-30), the need for more efficient and versatile delivery methods remains. For instance, AAV trans-splicing methods require high-dose AAV injections to compensate for their low efficiency and the random configuration of recombination events. This problem becomes more pronounced when more than two AAV vectors are needed. Similarly, the reconstitution efficiency of REVeRT appears to decrease significantly when the cargo gene is split into three AAV vectors. The split intein-based reconstitution approach entails the production of truncated proteins, which could potentially have dominant negative effects. Furthermore, the efficiency of protein splicing is heavily affected by the amino acids immediately adjacent to split inteins (25, 31, 32), and the impact of truncation on the folding and stability of both truncated protein products and reconstituted proteins is hard to predict. Due to these constraints, determining optimal splitting positions requires a screening process, and suitable positions may not always be available.

### SUMMARY

Adeno-associated virus (AAV) is a safe and efficient gene delivery vehicle for gene therapies. However, its relatively small packaging capacity limits its use as a gene transfer vector. Herein is described a strategy to deliver large genes that exceed the AAV's packaging capacity using up to four AAV vectors and the CRE-lox DNA recombination system. Novel lox sites were devised by combining non-compatible and reaction equilibrium-modifying lox site variants. These lox sites facilitate sequence-specific and near unidirectional recombination of AAV vector genomes, enabling efficient reconstitution of up to 16 kb of therapeutic genes in a pre-determined configuration. Using this strategy, AAV gene therapy vectors can be used to deliver, for example, IFT140, PCDH15, CEP290, and CDH23, and their efficient production of full-length proteins in cultured mammalian cells and mouse retinas was demonstrated. Notably, AAV-IFT140 gene therapy vectors ameliorated retinal degeneration and preserved visual functions in an IFT140-associated retinitis pigmentosa mouse model. The CRE-lox approach described here provides a simple, flexible, and effective platform for generating AAV gene therapy vectors beyond AAV's packaging capacity.

In one embodiment, an adeno-associated virus vector set for delivering a gene to a target cell comprises at least two recombinant AAV vectors, each flanked by AAV inverted terminal repeats and carrying a distinct fragment of the coding sequence. Adjacent fragments are bordered by matched hybrid lox site pairs, each hybrid lox site having an inverted repeat element mutated in only one of the left or right arms and a non-compatible spacer sequence that prevents recombination with any lox site other than its partner. The vector set may further include a promoter operably linked to one fragment (for example, a human cytomegalovirus immediate-early promoter or a rod photoreceptor-specific rhodopsin kinase promoter) and a transcription termination signal downstream of the final fragment (optionally comprising a polyadenylation signal). In certain configurations, the inverted-repeat mutation is confined to the left arm, the spacer sequence corresponds to lox2272, and each pair comprises a loxJT15 site and a loxJTZ17 site. The set may consist of two, three, or four or more vectors, one of which can also encode CRE recombinase downstream of the hybrid lox site, optionally operably linked to a T2A self-cleaving peptide. The gene of interest may encode human intraflagellar transport protein 140 (IFT140), PCDH15, CEP290, or CDH23 protein.

In another embodiment, a method to reconstitute a fragmented coding sequence in a target cell comprises co-administering at least two of these recombinant AAV vectors, each carrying a distinct fragment and a hybrid lox site; expressing CRE recombinase in the cell; and permitting unidirectional, sequence-specific recombination between matched hybrid lox site pairs to assemble the fragments into a contiguous, full-length coding sequence in a predetermined order. The vector set used in this method may consist of two, three, four or more vectors, may include a T2A-linked CRE recombinase cassette in one vector, and may incorporate the same promoter and termination signal options described above.

In a further embodiment, a method of treating or preventing a disease, disorder or cancer comprises administering the AAV vector set to a subject in need thereof. In one application, the disorder is a form of retinal degeneration, and the vector set carries a fragmented IFT140 gene that, upon recombination, yields expression of full-length IFT140 protein in the subject.

### BRIEF DESCRIPTION OF THE DRAWINGS

FIGS. 1A-1C. Lox site variants that enable CRE-dependent splicing of multiple AAV vector genomes. (A) Non-compatible mutant variants of loxP. Sequence differences (red) in the spacer region prevent recombination between non-compatible lox sites. Left and right elements (LE and RE, respectively) are palindromic. (SEQ ID NOs: 1-6) (B) Reaction equilibrium-modifying variants of loxP. LoxJT15 and loxJTZ17 sites have mutations (underlined) in either LE or RE but not in both. Recombination between two loxP sites (left) is fully reversible because substrates and products are identical. In contrast, recombination between loxJT15 and loxJTZ17 produces an LE/RE double mutant (lox15/17) and a loxP sequence. LE/RE double mutants are poor substrates for CRE, and consequently, the reverse reaction is significantly slower than the forward reaction. (SEQ ID NOs: 1 and 7-9) (C) Lox site variants that prevent recombination between non-compatible lox sites and inhibit reverse reactions. The spacer sequences of loxJT15 and loxJTZ17, which are from loxP, are replaced with those of the non-compatible lox sites (loxN, lox2272, loxm7 (not shown), loxHT1, and loxHT2). (SEQ ID NOs: 10-25)

FIGS. 2A-2B. Assessment of incompatibility among hybrid lox sites. (A) Schematics of reporter constructs to detect recombination events between loxJT15 (15:P), loxJTZ17:m7 (17:m7), loxJTZ17:HT1 (17:HT1), loxJTZ17:HT2 (17:HT2), loxJTZ17:2272 (17:2272), and loxJTZ17:N (17:N). The names of the reporter constructs (loxP-2272, loxP-N, lox2272-N, loxP-HT2, and lox2272-HT2) are shown on the left. Black hexagons denote stop codons. C290C: a 156-bp fragment from human CEP290 C-terminus (aa 2428-2479). (B) The spacers of loxP and lox2272 are fully incompatible with each other and with those of loxm7, loxHT1, and loxHT2. Reporter constructs shown in (A) were transfected to HEK293T cells with and without a CRE expression vector, and cell lysates were subjected to SDS-PAGE and immunoblotting. C290-C, FLAG, HA, V5, MYC, and b-actin antibodies were used for immunoblotting. A lysate derived from untransfected cells served as the negative control (lane 6), while lysates obtained from cells transfected with MYC-BBS1, FLAG-LZTFL1, and HA-LZTFL1 expression vectors were used as the positive control (lane 12). b-actin was used as a loading control. Numbers on the right of each panel indicate the locations of protein size markers.

FIGS. 3A-3B. CRE-lox-mediated reconstitution of large genes delivered by tripartite AAV vectors: proof-of-concept. (A) Schematic representation of large gene reconstitution by CRE-lox mediated recombination of three AAV vector genomes. A gene-of-interest is split into three fragments (CDS1, 2, and 3) and delivered to target cells via three separate AAV vectors. For a proof-of-concept demonstration, IFT140-N (5′ 1,923 bp of IFT140), BBS1, and LZTFL1 coding sequences were used as CDS1, 2, and 3, respectively. HA tag sequences were added to the 5′ ends of IFT140-N and BBS1. CRE recombinase was delivered via a separate AAV vector. SD: splice donor site, SA: splice acceptor site, 15: loxJT15, 17: loxJTZ17, and pA: polyA signal. (B) Production of IFT140N+BBS1+LZTFL1 fusion proteins (red arrowheads) from a tripartite AAV vector set. AAV vectors depicted in panel (A) were transduced to 293T cells, and the expression of IFT140N+BBS1+LZTFL1 fusion proteins was examined by SDS-PAGE and immunoblotting using HA and LZTFL1 antibodies. Numbers on the right mark the locations of protein standards. A separate AAV vector, AAV-EF1a-CRE, was co-transduced to express CRE (lanes 1-7). Lane 1: CDS1 (IFT140-N) only, lane 2: CDS2 (BBS1) only, lane 3: CDS3 (LZTFL1) only, lane 4: CDS1+CDS2, lane 5: CDS1+CDS3, lane 6: CDS2+CDS3, lane 7: CDS1+CDS2+CDS3, lane 8: CDS1+CDS2+CDS3 (without CRE). Endogenous LZTFL1 (blue arrowheads) was used as a loading control.

FIGS. 4A-4B. CRE-lox-mediated reconstitution of large genes delivered by quadripartite AAV vectors: proof-of-concept. (A) Schematic representation of large gene reconstitution by CRE-lox mediated recombination of four AAV vector genomes. A gene-of-interest is split into four fragments (CDS1, 2, 3, and 4) and delivered to target cells via four separate AAV vectors. For a proof-of-concept demonstration, IFT140-N (5′ 1,923 bp of IFT140), IFT57, BBS5, and LZTFL1 coding sequences were used as CDS1, 2, 3, and 4, respectively. HA tag sequences were added to the 5′ ends of IFT140-N and BBS5. Others are the same as in FIG. 3. (B) Production of IFT140N+IFT57+BBS5+LZTFL1 fusion proteins (red arrowheads) from a quadripartite AAV vector set. AAV vectors depicted in panel (A) were transduced to 293T cells, and the expression of IFT140N+IFT57+BBS5+LZTFL1 fusion proteins was examined by SDS-PAGE and immunoblotting using HA and LZTFL1 antibodies. Endogenous LZTFL1 (blue arrowheads) was used as a loading control. Others are the same as in FIG. 3.

FIGS. 5A-5H. Reconstitution of IFT140 by CRE-lox-mediated recombination. (A) Schematic representation of IFT140 reconstitution by CRE-lox-mediated recombination of bipartite AAV vector genomes. T2A: T2A “self-cleaving” peptide, IRES: internal ribosome entry site. Others are the same as in FIG. 3. (B) Production of full-length IFT140 proteins (red arrowheads) through CRE-lox-mediated recombination in HEK293T cells. HEK293T cells were transduced with dual AAV vectors depicted in panel A (with a CMV promoter), and cell lysates were subjected to SDS-PAGE followed by immunoblotting with HA and IFT140-C antibodies. Lane 1: AAV-IFT140_N only, lane 2: AAV-IFT140_C only, lane 3: dual AAV-IFT140_N+C. b-actin was used as a loading control. (C) Expression of full-length IFT140 in normal mouse retinas using the dual AAV-IFT140 CRE/lox set. Dual AAV-GRK1p-IF7740 vectors were administered via subretinal injection into mouse eyes (serotype: Anc80L65, dose: 8×108 GC per vector), and eyes were collected 2 weeks post-injection for immunoblotting with HA, IFT140-C, and CRE antibodies. Lanes 1-2: uninjected (n=2), lanes 3-5: AAVIFT140_N only (n=3), lanes 6-8: AAV-IFT140_C only (n=3), and lanes 9-12: AAV-IFT140_N+C (n=4). Each lane corresponds to an individual eye. The blue and black arrowheads indicate the IFT140-N product and CRE, respectively. (D) Reconstitution of IFT140 expression in Ift140 CKO mice using dual AAV-GRK1p-IFT140 vectors. Dual AAV-GRK1p-IFT140 CRE/lox vectors were delivered to the subretinal space of normal (Ift140fl/fl;iCre75−) and Ift140 CKO (Ift140fl/fl;iCre75+) mice at P16, and IFT140 expression was analyzed by immunoblotting at P55. Both injected and uninjected eyes were collected, and their protein extracts were loaded side-by-side (indicated by black lines). Each lane corresponds to an individual eye. (E, F) Preservation of photoreceptor cells in Ift140 CKO mice by dual AAV-GRK1p-IFT140 CRE/lox vectors. Dual AAV-GRK1p-IFT140 CRE/lox vectors were delivered to the subretinal space of normal and Ift140 CKO mice at P16, and retinal histology was examined by immunohistochemistry using HA, PRPH2, and GNAT2 antibodies at P52. Nuclei were counterstained with DAPI. Brackets delineate the outer nuclear layer (ONL). Scale bar: 50 μm. INL: inner nuclear layer. (G, H) Preservation of retinal function in Ift140 CKO mice by IFT140 subretinal gene therapy. Normal (n=6) and Ift140 CKO (n=6) mice were administered dual AAV-GRK1p-IFT140 vectors in one eye at P16, and rod (G) and cone (H) functions were evaluated by scotopic and photopic ERG at P45. Uninjected contralateral eyes were used as controls. Rod function was measured using 0.01 cd·s/m² dim flashes after dark adaptation, while cone function was measured using 3.0 cd·s/m² bright flashes after light adaptation. ERG b-wave amplitudes (mean±SD) are shown in the graphs. Asterisks indicate statistical significance (two-tailed Student's t-test; p<0.05). ns: not significant.

FIGS. 6A-6B. Reconstitution of PCDH15 using the CRE-lox recombination approach. (A) Strategy for the PCDH15 reconstitution using dual AAV-PCDH15 vectors. PCDH15 CDS was divided into two segments (1,932 bp and 3,933 bp) for delivery, and a FLAG tag (red) sequence was added to the 3′ end of the gene. PCDH15-N antibody was raised against the N-terminal half of human PCDH15 (aaQ27-A1376). Others are the same as in FIG. 3. (B) Reconstitution of PCDH15 by dual AAV-PCHD15 CRE-lox vectors. HEK293T cells were transduced with dual AAV2/2-CMV-PCHD15 CRE-lox vectors as indicated, and cell lysates were subjected to SDSPAGE and immunoblotting with PCDH15-N and FLAG antibodies. Red arrowheads mark the reconstituted full-length PCDH15 proteins.

FIGS. 7A-7D. CRE-lox mediated reconstitution of CEP290. (A) Schematic representation of the bipartite AAV-(EP290 vectors. The 5′ vector is composed of a CMV promoter, 5′ 3,527 bp of human CEP290, an SD site, and a loxJT15 site. The 3′ vector consists of a loxJTZ17 site, an SA site, 3′ 3,913 bp of CEP290, and a BGH polyA signal. A FLAG tag was added to the 5′ end of CEP290. Destabilizing domain (DD)-fused CRE was delivered via a separate AAV vector (AAV-EF1a-DD-CRE). The location of the C290-C antibody epitope was marked by a black line at the bottom of the schematic. (B) Production of full-length CEP290 proteins (red arrowhead) by CRE-lox-mediated recombination in 293T cells. HEK293T cells were transduced with dual AAV2/2-CEP290 (MOI: 3×104 GC/cell per vector) and AAV2/2-EF1a-DD-CRE (MOI: 1×104 GC/cell) vectors. After transduction, cells were treated with 10 μM trimethoprim (TMP) for 48 hours to stabilize DD-CRE, and cell lysates were subjected to SDSPAGE followed by immunoblotting with FLAG and C290-C antibodies. Lane 1: AAV-CEP290_N only, lane 2: AAV-CEP290_C only, lanes 3-4: dual AAV-CEP290_N+C, and lane 5: pSS-FS-hCEP290 plasmid transfected (full-length; positive control). Cells in lanes 1-3 were co-transduced with AAV2/2-EF1a-DD-CRE. Numbers on the right denote the location of protein standards. b-actin was used as a loading control. (C) Schematic representation of the tripartite AAV-CEP290 vectors. The 5′ vector (E1) is composed of a CMV promoter, 5′ 1,065 bp of human CEP290, an SD site, a loxJT15 site, IRES, CRE CDS, and a BGH poly A signal. The middle vector (E2) is composed of a loxJTZ17 site, an SA site, 2,913 bp of CEP290 CDS, an SD site, and a lox15:2272 site. The 3′ vector (E3) consists of a lox17:2272 site, an SA site, 3′ 3,462 bp of CEP290, and a BGH poly A signal. Others are the same as in panel A. (D) Production of full-length CEP290 proteins (red arrowhead) by CRE-lox-mediated recombination in 293T cells. Others are the same as in panel B.

FIGS. 8A-8C. CRE-lox-mediated reconstitution of CDH23 delivered by tripartite AAV vectors. (A) Schematic of the CDH23 reconstitution using tripartite AAV-CDH23 vectors. The CDH23 CDS (10,065 bp) was split into three pieces (E1: 2,176 bp, E2: 4,077 bp, and E3: 3,812 bp), and the CRE gene was included in the 5′ (E1) vector for self-inactivation after recombination. A T2A “self-cleaving” peptide was used for CRE expression. An HA tag (red) was added to the N-terminus of CDH23 for detection (after the signal peptide). (B) Production of full-length CDH23 proteins (red arrowhead) by CRE-lox-mediated recombination in 293T cells. HEK293T cells were transduced with tripartite AAV2/2-CDH23 vectors (MOI: 3×104 GC/cell of each vector), and cell lysates were subjected to SDS-PAGE followed by immunoblotting with HA antibodies. Lane 1: AAV-CDH23-E1 only, lane 2: AAV-CDH23-E2 only, lane 3: AAV-CDH23-E3 only, lane 4: AAV-CDH23-E1, E2, and E3 co-transduced, and lane 5: pSS-HA-CDH23-SF plasmid transfected (full-length; positive control). b-actin was used as a loading control. (C) Expression of CDH23 from the tripartite AAV-CDH23 vectors in mouse retinas. Tripartite AAVCDH23 vectors were subretinally administered to wild-type mice as indicated (lane 1: E1 vector alone, lane 2: E2 vector alone, lane 3: E3 vector alone, lanes 4-7: E1, E2, and E3 co-injected) at the dose of 3×109 GC per vector (n=3-4 per vector or vector set). Treated eyes were collected 3 weeks post-injection and retinal protein extracts were subjected to SDS-PAGE and immunoblotting. Each lane represents an individual eye.

FIGS. S1A-S1E. When there are two or more loxP sites in a single DNA molecule, the intervening “floxed” sequence is rapidly excised (S1A). If all DNA fragments have the same lox sites, recombination can occur in any combination (S1B), preventing the specification of the order of DNA segments in end products, leading to the production of unintended, non-functional products. Lastly, since the CRE-lox recombination is fully reversible, the reconstituted DNA constantly goes through the assembly-disassembly cycle, limiting the yield of the reconstituted DNAs. If the=hybrid lox sites are fully non-compatible with one another, they will prevent the excision of intervening sequences and unintended recombinations (S1C and S1D). At the same time, they will significantly increase the yield of reconstituted DNA products by inhibiting reverse reactions, particularly when using three or more AAV vectors (S1E).

FIG. S2. To assess the compatibility of the developed hybrid lox sites in mammalian cells, GFP expression cassettes capable of tracking recombination events between different lox sites were developed.

FIGS. S3 and S4. Retinal degeneration is noticeable at P24 and progresses rapidly in Ift140/11;iCre75⁺ mice. To prevent constitutive overexpression of CRE, the dihydrofolate reductase destabilizing domain (DD)-fused CRE was adopted.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Abbreviations

AAV: adeno-associated virus
BGH: bovine growth hormone
CDS: coding sequence
DD: destabilizing domain
GC: genome copies
HRP: horse radish peroxidase
IntC: C-terminal split intein
IntN: N-terminal split intein
IRES: internal ribosome entry site
ITR: inverted terminal repeat
LCA: Leber congenital amaurosis
LE: left element
MOI: multiplicity of infection
RE: right element
RP: retinitis pigmentosa
SA: splice acceptor
SD: splice donor
TMP: trimethoprim

Gene therapy has emerged as a promising approach for treating genetic disorders, leveraging the ability of viral vectors, such as adeno-associated viruses (AAVs), to deliver therapeutic genes to target cells. However, a significant limitation of AAV vectors is their relatively small packaging capacity, which is approximately 4.7 to 5 kilobases (kb). This constraint precludes the delivery of many large therapeutic genes, such as IFT140, CEP290, PCDH15, and CDH23, which are implicated in severe genetic disorders, including retinal degeneration and Usher syndrome. Existing strategies to overcome this limitation, such as dual or triple AAV vector systems, rely on methods like trans-splicing, overlapping sequences, or protein trans-splicing using split inteins. While these approaches have demonstrated some success, they suffer from inefficiencies, including low reconstitution yields, random recombination configurations, truncated protein products with potential dominant-negative effects, and the need for high vector doses to achieve therapeutic efficacy. These limitations hinder the scalability, safety, and effectiveness of gene therapies for large genes.

The present system addresses these challenges by introducing a novel approach for delivering large therapeutic genes using up to four AAV vectors in conjunction with the CRE-lox DNA recombination system. This system employs specially engineered lox site embodiments that combine non-compatible and reaction equilibrium-modifying properties. These hybrid lox sites enable sequence-specific and near-unidirectional recombination of AAV vector genomes, ensuring efficient and precise reconstitution of therapeutic genes in a predetermined configuration. This approach improves upon prior methods by enhancing recombination efficiency, reducing the production of truncated or unintended protein products, and minimizing the need for high vector doses. Furthermore, the inclusion of the CRE recombinase gene within the AAV vectors allows for self-contained and self-limiting recombination, reducing the risk of prolonged CRE expression and associated genomic instability.

By leveraging these innovations, the described platform provides a flexible and scalable approach for delivering large genes, enabling the development of effective gene therapies for a wide range of genetic disorders. The system has been successfully demonstrated in the delivery of genes such as IFT140, PCDH15, CEP290, and CDH23, with therapeutic efficacy validated in preclinical models, including the preservation of retinal function in an IFT140-associated retinitis pigmentosa mouse model. This solution represents a significant advancement in addressing the packaging limitations of AAV vectors, offering a robust and versatile framework for addressing unmet medical needs in gene therapy.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N. Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of.” “only one of,” or “exactly one of”.

As used herein, the terms “including,” “includes,” “having.” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “regulatory element” is meant to include promoters, enhancers, internal Ribosome Entry Sites (IRES) and other expression control elements (e.g., transcription termination signals such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990).

Sequence homology can be generated by any of a number of computer programs known in the art, such as BLAST or FASTA, and the like. A suitable computer program for performing such an alignment is GCG Wisconsin Bestfit software package (University of Wisconsin, U.S.). Examples of other software that may perform sequence comparisons include, but are not limited to, BLAST packages (see Ausubel et al, 1999 supra, chapter 18), FASTA (Atschul et al, 1990, J. Mol. Biol., 403-410), and GENEWORKS comparison kits. Both BLAST and FASTA can be used for both offline and online searches (see Ausubel et al, 1999 supra, pages 7-58 to 7-60). The percent (%) sequence homology can be calculated over consecutive sequences, i.e., one sequence is aligned with another sequence and each amino acid or nucleotide in one sequence is directly compared to the corresponding amino acid or nucleotide in the other sequence, one residue at a time. This is referred to as a “vacancy free” alignment. Typically, such vacancy free alignments are performed on only a relatively small number of residues. Although this is a very simple and consistent method, it does not take into account that, for example, in otherwise identical pairs of sequences, an insertion or deletion may cause subsequent amino acid residue alignments to be unsuccessful, which may result in a substantial reduction in the percentage of homology when global alignments are performed. Thus, most sequence comparison methods are designed to produce optimal alignments that take into account possible insertions and deletions without undue penalty for overall homology or identity scores. This is accomplished by inserting “gaps” in the sequence alignment in an attempt to maximize local homology or identity. However, these more complex methods assign a “gap penalty” to each gap that occurs in an alignment, so that for the same number of identical amino acids, sequence alignment with as few gaps as possible (reflecting a higher correlation between the two compared sequences) can achieve a higher score than sequence alignment with many gaps. An “affinity gap cost” (AFFINITY GAP cost) is typically used, which charges a relatively high cost for the presence of a gap, and a small penalty for each subsequent residue in the gap. This is the most commonly used vacancy scoring system. Of course, high gap penalties can result in optimized alignments with fewer gaps. Most alignment programs allow for modification of the gap penalty. But it is preferred to use default values when using such software for sequence comparison. For example, when using the GCG Wisconsin Bestfit software package, the default gap penalty for amino acid sequences is-12 for gaps and-4 for each extension. Thus, calculating the maximum percent homology first requires that an optimal alignment be produced taking into account the gap penalty. Although the final percent homology can be measured in terms of identity, the alignment process itself is typically not based on a full pairing or no pairing comparison. Instead, a proportional similarity score matrix is typically used that assigns a score to each pair-wise comparison based on chemical similarity or evolutionary distance. An example of such a matrix that is commonly used is the BLOSUM62 matrix, which is the default matrix for the BLAST suite of programs. The GCG Wisconsin program, if provided, typically uses a common default or custom symbol comparison table (see user manual for more details). For some applications it is preferred to use a common default value for the GCG package, or in the case of other software, it is preferred to use a default matrix such as BLOSUM62. Alternatively, the percent homology can be calculated using multiple alignment features in DNASIS™ (Hitachi Software) based on an algorithm similar to CLUSTAL (HIGGINS D G & Sharp P M (1988), gene 73 (1), 237-244).

Once the software produces the optimal alignment, the percent homology/percent sequence identity, can be calculated. The software typically takes this as part of the sequence comparison and generates a numerical result. These sequences may also have deletions, insertions or substitutions of amino acid residues which produce a silent change and form a functionally equivalent substance. Intentional amino acid substitutions may be made based on similarity in amino acid properties such as polarity, charge, solubility, hydrophobicity, hydrophilicity, and/or the amphipathic nature of the residues, and thus grouping amino acids together according to functional groups is useful. Amino acids may be grouped together based solely on the nature of the amino acid side chains. But it may be more useful to include mutation data as well. The resulting collection of amino acids may be conserved for structural reasons.

As used herein, the term “variant” is understood to mean exhibiting a quality that differs from the naturally occurring pattern. The terms “non-naturally occurring” or “engineered” are used interchangeably to refer to human intervention.

The terms “subject,” “individual,” and “patient” are used interchangeably herein to refer to a vertebrate, such as a mammal, including a human. Mammals include, but are not limited to, mice, apes, humans, farm animals, sports animals, and pets. Tissues, cells, and progeny thereof of the biological entity obtained in vivo or cultured in vitro are also included.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “substantially homologous amino acid sequences” includes those amino acid or nucleic acid sequences which have at least about 95% homology, at least about 96% homology, at least about 97% homology, at least about 98% homology, or at least about 99% or more homology to an amino acid or nucleic sequence of a reference.

The term “amino acid” is used interchangeably with “amino acid residue,” and may refer to a free amino acid and to an amino acid residue of a peptide/protein. It will be apparent from the context in which the term is used whether it refers to a free amino acid or a residue of a peptide/protein.

The expression “amino acid” as used herein is meant to include both natural and synthetic amino acids, and both D and L amino acids. “Standard amino acid” means any of the twenty standard L-amino acids commonly found in naturally occurring peptides/proteins. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides/proteins of the present invention, and particularly at the carboxy- or amino-terminus, can be modified by methylation, amidation, acetylation or substitution with other chemical groups which can change the peptide's/protein's circulating half-life without adversely affecting their activity (e.g., peptidomimetic for making peptides protease resistant).

Amino acids have the following general structure:

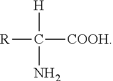

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide/protein compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met, Leu, Ile, Val, Cys
V. Large, aromatic residues:
   Phe, Tyr, Trp.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

### Adeno-Associated Virus (AAV)

Although adeno-associated virus (AAV) is a safe and efficient gene delivery vehicle for gene therapy, previously described methods to deliver large genes using dual or triple AAV vectors have notable limitations in their use. These methods include hybrid dual AAV and split intein-mediated protein trans-splicing approaches (Ghosh et al, 2008; Tornabene et al, 2019; Trapani et al, 2021; Villiger et al, 2018). For instance, the hybrid dual AAV approach suffers from very low reconstitution efficiency. Meanwhile, the efficiency and use of the split intein-mediated protein trans-splicing approach is affected by several factors: 1) the amino acid residues at the split site, which affect protein trans-splicing efficiency, 2) the stability and structure of the expressed N- and C-terminal truncated protein products, 3) the subcellular localization of these truncated protein products (co-localization is necessary for trans-splicing but may not occur), and 4) the potential for detrimental effects from the truncated protein products.

Provided herein is a novel strategy to deliver large genes using up to four AAV vectors. Cargo genes are split into 2-4 AAV vectors and reconstituted by using the CRE-lox DNA recombination system. The use of novel lox sites, which were generated by combining non-compatible and reaction equilibrium-modifying lox site variants, enables efficient reconstitution of a therapeutic cassette in a predetermined configuration.

As a proof of principle and therapeutic compositions and methods, provided herein are adeno-associated virus (AAV)-based gene therapy vectors to deliver the full-length IFT140 gene and treat retinal degeneration caused by IFT140 mutations.

Provided herein is a method for delivering the full-length IFT140 gene to affected tissues using dual AAV vectors and reconstitute via the CRE-lox site-specific DNA recombination system. To enhance reconstitution efficiency, novel lox sites were designed by combining non-compatible and reaction equilibrium-modifying lox site variants. These hybrid lox sites enable sequence-specific and near-unidirectional recombination of AAV vector genomes in a pre-determined configuration. Using this approach, gene therapy vectors were developed that efficiently deliver the full-length IFT140 gene through dual AAV vectors. It is further demonstrated that these vectors successfully produce full-length IFT140 proteins, improve visual function, and delay or prevent retinal degeneration in Ift140 mutant mice.

### AAV

In some aspects, the vectors are AAV vectors. In some aspects, the vectors are single stranded AAV vectors. In some aspects the AAV is recombinant AAV (rAAV). In some aspects, the rAAV lack rep and cap genes. In some aspects, rAAV are self-complementary (sc) AAV. There are multiple serotypes of AAV. The nucleotide sequences of the genomes of the AAV serotypes are known. For example, the complete genome of AAV-1 is provided in GenBank Accession No. NC 002077; the complete genome of AAV-2 is provided in GenBank Accession No. NC 001401 and Srivastava et al., J. Virol., 45:555-564 11983); the complete genome of AAV-3 is provided in GenBank Accession No. NC 1829; the complete genome of AAV-4 is provided in GenBank Accession No. NC 001829; the AAV-5 genome is provided in GenBank Accession No. AF085716; the complete genome of AAV-6 is provided in GenBank Accession No. NC 00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78:6381-6388 (2004); the AAV-10 genome is provided in Mol. Ther., 13(1): 67-76 (2006); and the AAV-11 genome is provided in Virology, 330(2): 375-383 (2004). AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh. 74. As set out herein above, the nucleotide sequences of the genomes of various AAV serotypes are known in the art.

Recombinant expression vectors may comprise a nucleic acid of the invention in a form suitable for expression of the nucleic acid in a host cell, which means that the recombinant expression vector comprises one or more regulatory elements operably linked to the nucleic acid sequence to be expressed, which regulatory elements may be selected according to the host cell to be used for expression. Within a recombinant expression vector, “operably linked” refers to the linkage of a nucleotide sequence of interest to one or more other elements in a manner that allows for expression of the nucleotide sequence (e.g., in an in vitro transcription/translation system, or in a host cell when the vector is introduced into the host cell).

One or more vectors may include one or more regulatory elements, such as one or more promoters.

### Genes of Interest

Any gene or coding sequence may be fragmented and recombined using the compositions and methods disclosed herein to express a functional protein. Exemplary genes include, but are not limited to, IFT140, PCDH15, CEP290, and CDH23, such as human IFT140 (BC035577), human PCDH15 (NM_033056.4), human CEP290 CDS (NM_025114.4) or human CDH23 (NM_022124.6) coding sequences.

IFT140, a component of the intraflagellar transport (IFT) system, plays a role in maintaining the structure and function of photoreceptor cells. Mutations in IFT140 cause inherited retinal dystrophy and short-rib thoracic dysplasia in humans (Hull et al, 2016; Perrault et al, 2012; Xu et al, 2015). Despite the devastating impact these conditions have on patients' quality of life, no treatment has been developed to delay or prevent vision loss, partly because of the gene's relatively large size.

PCDH15 stands for “protocadherin-15,” a protein-coding gene belonging to the cadherin superfamily. This protein functions as a calcium-dependent cell adhesion molecule and plays a role in the inner ear, specifically in hair cell stereocilia, where it forms part of the tip-link filaments essential for mechanosensory transduction (hearing). It also plays a role the maintenance of normal retinal function. Mutations in PCDH15 are linked to hearing loss and Usher Syndrome type IF, which features deafness, balance problems, and progressive blindness.

CEP290 is a gene that encodes the centrosomal protein of 290 kDa, which plays a role in centrosome and cilia development in human cells. This protein is particularly important for the formation and function of primary cilia-small, antenna-like projections on the cell membrane that play roles in sensory perception, especially vision. In the retina, CEP290 helps transport proteins within photoreceptor cells and is necessary for normal vision. Mutations in CEP290 are associated with several genetic diseases, most notably Leber congenital amaurosis (LCA), a severe early-onset form of blindness, as well as other ciliopathies like Joubert syndrome and Meckel syndrome. These conditions are related to abnormal formation or function of cilia due to defective CEP290 protein.

CDH23 refers to the gene encoding cadherin 23, a calcium-dependent cell adhesion protein. Cadherin 23 is part of the cadherin superfamily of glycoproteins that help cells stick together, and it plays a role in organizing and maintaining the structure of stereocilia-hair-like projections in the inner ear. These stereocilia are needed for converting sound waves into nerve signals for hearing and also play a role in balance. Mutations in CDH23 are linked to nonsyndromic hearing loss (specifically DFNB12) and Usher syndrome type ID, which is characterized by congenital deafness and progressive vision loss. The protein is also expressed in the retina.

### Treatment/Administration

The invention also includes methods for delivering one or more nucleic acid components to a cell and/or a subject. In certain embodiments, the cell is a eukaryotic cell, such as mammalian cell, including a human cell.

In some embodiments, the vector, e.g., plasmid or viral vector, is delivered to the target tissue by, e.g., intramuscular injection, while other times are delivered via intravenous, transdermal, intranasal, oral, mucosal, or other delivery methods. Such delivery may be via a single dose or multiple doses. It will be appreciated by those skilled in the art that the actual dosage delivered herein may vary greatly depending on a variety of factors, such as carrier selection, target cells, organisms or tissues, general condition of the subject to be treated, degree of transformation/modification sought, route of administration, mode of administration, type of transformation/modification sought, and the like.

In one embodiment herein, delivery is via AAV. A therapeutically effective dose for in vivo delivery of AAV to a human is believed to range from about 20 to about 50 ml of saline solution containing from about 1×10¹⁰ to about 1×10¹⁰ functional AAV/ml of solution. Dosages may be adjusted to balance therapeutic benefit with any side effects. In one embodiment herein, the concentration of AAV dose is typically in the range of about 1×10⁵ to 1×10⁵⁰ genomic AAV, about 1×10⁸ to 1×10²⁰ genomic AAV, about 1×10¹⁰ to about 1×10¹⁶ genomic, or about 1×10¹¹ to about 1×10¹⁶ genomic AAV. The human dose may be about 1×10¹³ genomic AAV. Such concentrations may be delivered in about 0.001 ml to about 100 ml, about 0.05 to about 50 ml, or about 10 to about 25 ml of carrier solution. Other effective dosages can be readily determined by one of ordinary skill in the art by routine experimentation to establish a dose response curve.

Such dosages may also include, for example, a carrier (water, saline, ethanol, glycerol, lactose, sucrose, calcium phosphate, gelatin, dextran, agar, pectin, peanut oil, sesame oil, etc.), a diluent, a pharmaceutically acceptable carrier (e.g., phosphate buffered saline), a pharmaceutically acceptable excipient, and/or other compounds known in the art. The dose may also comprise one or more pharmaceutically acceptable salts, such as mineral acid salts, such as hydrochloride, hydrobromide, phosphate, sulfate, and the like; and salts of organic acids such as acetates, propionates, malonates, benzoates, etc. In addition, auxiliary substances such as wetting or emulsifying agents, pH buffering substances, gels or jellifying materials, flavoring agents, coloring agents, microspheres, polymers, suspending agents, and the like may also be present therein. In addition, one or more other conventional pharmaceutical ingredients may also be present, such as preservatives, wetting agents, suspending agents, surfactants, antioxidants, anti-caking agents, fillers, chelating agents, coating agents, chemical stabilizers, and the like, especially if the dosage form is of a reconfigurable form. Suitable exemplary ingredients include microcrystalline cellulose, sodium carboxymethyl cellulose, polysorbate 80, phenethyl alcohol, chlorobutanol, potassium sorbate, sorbic acid, sulfur dioxide, propyl gallate, parabens, ethyl vanillin, glycerin, phenol, p-chlorophenol, gelatin, albumin, and combinations thereof. A detailed discussion of pharmaceutically acceptable excipients is available in REMINGTON'S PHARMACEUTICAL SCIENCES (Mack Pub.Co., N.J. 1991), which is incorporated herein by reference.

The following Example illustrates some of the materials, methods, and experiments that were used or performed in the development of the invention.

### EXAMPLE

### Introduction

AAV gene therapy vectors were developed to deliver large genes, including but not limited to, IFT140, PCDH15, CEP290, and CDH23, and their efficient production of full-length proteins in mammals, as demonstrated in cultured mammalian cells and mouse retinas. Notably, AAV-IFT140 gene therapy vectors ameliorated retinal degeneration and preserved visual functions in an IFT140-associated retinitis pigmentosa mouse model. The CRE-lox approach described here provides a simple, flexible, and effective platform for generating AAV gene therapy vectors beyond AAV's packaging capacity.

### Materials and Methods

### Plasmid DNAs

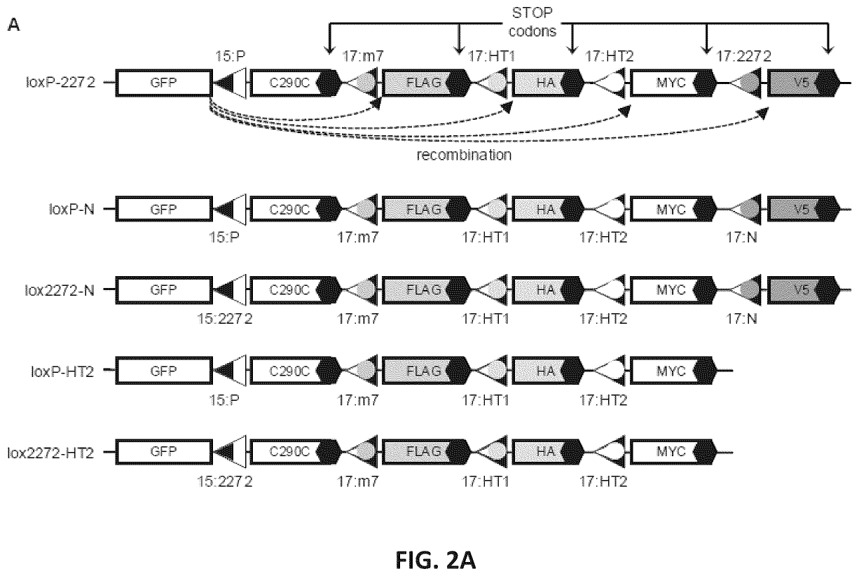

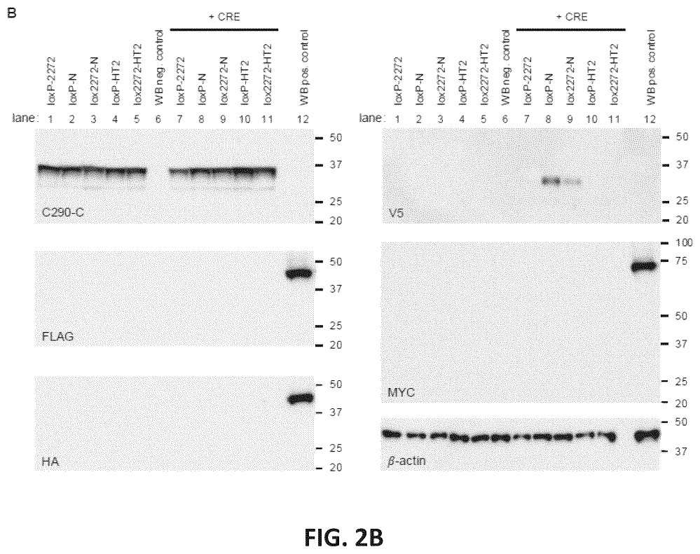

To generate the GFP expression reporter loxP-N (FIG. 2), a double-stranded DNA fragment corresponding to the loxJT15+C290C+17:m7+FLAG+17:HT1+HA+17:HT2+MYC+17:N+V5 portion was synthesized (Integrated DNA Technologies) and inserted at the C-terminus of GFP within the pEGFPC3 plasmid (Clontech) using a GenBuilder Cloning kit (GenScript). Reporter loxP-2272 was generated by substituting the lox17:N site (ATAACTTCGTATAGTATACCTTATAGCAATTTAT; SEQ ID NO: 26) within loxP-N with the lox17:2272 site (ATAACTTCGTATAGGATACTTTATAGCAATTTAT; SEQ ID NO: 27) using a Q5 Site-Directed Mutagenesis kit (New England Biolabs) and PCR. Reporter lox2272-N was produced by replacing the loxJT15 site (AATTATTCGTATAGCATACATTATACGAAGTTAT; SEQ ID NO: 28) in the loxP-N reporter with the lox15: 2272 site (AATTATTCGTATAGGATACTTTATACGAAGTTAT; SEQ ID NO: 29). Reporters loxP-HT2 and lox2272-HT2 were derived by eliminating the lox17:N sites in loxP-N and lox2272-N using a Q5 Site-Directed Mutagenesis kit and PCR. The DNA sequences downstream of GFP are shown.

Briefly, the pFBAAV-related plasmids were generated by modifying the pFBAAVCMVmcsBGHpA plasmid (from the Viral Vector Core Facility, University of Iowa). Plasmid pAAV-CBh-JT1522 was custom-synthesized and procured from VectorBuilder. Other pAAV-related plasmids were developed by modifying these plasmids using standard molecular biology techniques and the GenBuilder Cloning kit (GenScript). PCR primers and oligonucleotides used were obtained from Integrated DNA Technologies (IDT). Plasmids pcDNA3-MYC-hBBS1, pCS2FLAG-hLZTFL1, and pCS2HA-hLZTFL1 were previously described (59, 60). Plasmid DNAs containing human IFT140 (BC035577) and PCDH15 (NM_033056.4) CDSs were acquired from transOMIC and GenScript, respectively. These plasmids were used as PCR templates to produce pFBAAV-CMV-IFT140N-15, pAAV-CMV-IFT140N-1522-CRE, pFBAAV-1722-IFT140C-pA, pAAV-GRK1p-IFT140N-1522-CRE, pFBAAV-1722-IFT140C-pAv3, pAAV-CMV-PCDH1SN-15-IRES-CRE, and pFBAAV-17-PCDH15CpA. A plasmid containing the full-length human CEP290 CDS (NM_025114.4) was reported earlier (61) and used as a PCR template for bipartite (pFBAAV-CMV-CEP290N-JT15 and pFBAAV-17-CEP290CpA) and tripartite AAV-CEP290 vectors (pAAV-CBh-CEP290-E1-15-CRE, pAAV-17-CEP290-E2-1522, and pFBAAV-1722-CEP290-E3-pA). The human CDH23 CDS was divided into 5 segments and synthesized by IDT according to the NM_022124.6 sequence. An HA tag was introduced after CDH23's signal peptide (N-terminal 28 residues). These 5 segments were PCR-amplified using Q5 High-Fidelity DNA polymerase and inserted into the multiple cloning site (MCS) of pAAV-CBh-JT15-CREv2 (first segment), pAAV-JTZ17-mid-1522 (second and third segments), and pFBAAV-1722-pA (fourth and fifth segments) using the GenBuilder Cloning kit to produce pAAV-CBh-CDH23-E1-15-CRE, pAAV-17-CDH23-E2-1522, and pFBAAV-1722-CDH23-E3-pA, respectively. A full-length CDH23 expression vector, pSS-HA-hCDH23-SF, was generated by PCR amplification of the CDH23-E1, E2, and E3 fragments and insertion of these 3 fragments into the MCS of the pSS-SF plasmid (62) using the GenBuilder Cloning kit.

### Transfection and AAV Transduction in HEK293T/17 Cells

HEK293T/17 cells were acquired from American Type Culture Collection (ATCC; #CRL-11268) and cultured in Dulbecco's Modified Eagle's Medium (DMEM; Gibco) supplemented with 10% (v/v) feta bovine serum (FBS; Gibco) and 100 units/ml penicillin/streptomycin (Gibco) at 37° C. in a humidified 5% CO₂ incubator. Plasmid DNAs were transfected in 12-well plates (Sarstedt) using FuGENE H Transfection Reagent (Promega) following the manufacturer's instructions. Twenty-four hours post-transfection cells were transferred to 6-well plates and cultured for 48 hours before harvesting.

AAV vectors with serotype 2 were produced by VectorBuilder and used for transducing HEK293T/17 cells. Approximately 5×10⁴ cells/well were seeded in a 24-well plate (Sarstedt) 24 hours prior to transduction. On the day of transduction, AAV vectors were thawed on ice, and appropriate volumes were taken to achieve the desired MOI and added to 250 μl of DMEM with 2% FBS. After removing the existing culture medium, the AAV-containing medium was added to the cells. The cells were then incubated for 16-17 hours in the cell culture incubator. Transduced cells were expanded in 6-well plates with complete culture medium and further incubated for 72 hours before harvesting. For trimethoprim (TMP) treatment, transduced cells were expanded in 6-well plates with complete medium containing 10 μM TMP (Sigma), cultured for 48 hours, and then switched to a normal culture medium for an additional 24 hours before harvesting.

### Mouse, Animal Study Approval, and AAV Subretinal Injection

Wild-type C57BL/6J mice (strain #: 000664) were acquired from the Jackson laboratory. Ift140fl and iCre7 mice were generously provided by Dr. Gregory J. Pazour (University of Massachusetts) and Dr. Ching-Kang Chen (University of Texas Health Science Center at San Antonio), respectively (47-49). Genotyping was performed as previously described (47, 48, 61, 63)

All animal procedures were approved by the Institutional Animal Care and Use Committee (IACUC) of the University of Iowa and conducted in accordance with the recommendations outlined in the Guide for the Care and Use of Laboratory Animals of the National Institutes of Health. All animals were maintained in 12-hour light-dark cycles and fed standard mouse chow ad libitum.

For subretinal injections, AAV/Anc80-GRK1p-IFT140 vectors (L65 variant) were prepared by the Viral Vector Core Facility at the University of Iowa. AAV-CDH23 vectors were packaged in the AAV5 serotype and produced by VectorBuilder. On the day of injection, AAV vectors were thawed on ice and mixed/diluted to desired titers (8×10⁸ GC/μl per vector for AAV-IFT140 and 3×10⁹ GC/μl per vector for AAV-CDH23) in PBS supplemented with 0.001% Poloxamer 188 (Sigma Aldrich).

For the gene expression analysis in normal mice, 1-2-month-old wild-type C57BL/6J mice (both males and females) were used and at least 3 mice were injected per vector or vector set. For IFT140 subretinal gene therapy in Ift140 CKO mice, injections were administered at P16 in both males and females. Each animal received a subretinal injection in one eye, with the contralateral eye serving as an uninjected control. Mice were anesthetized with a ketamine/xylazine mixture (87.5 mg/kg ketamine, 12.5 mg/kg xylazine), and 10% povidone-iodine and 1% tropicamide solutions were applied. Under a Zeiss OPMI VISU 150 surgical microscope, eyes were gently rotated using forceps, and a conjunctival peritomy was made with Vannas scissors, followed by a sclerotomy made posterior to the limbus using a 30-gauge needle. Transscleral subretinal injections were performed with a Hamilton syringe attached to a blunt-end 32-gauge needle inserted subretinally at an oblique angle, delivering 1 μl of vector solution. After injections, an antibiotic/steroid ophthalmic ointment (neomycin and dexamethasone) was applied. Animals were excluded from follow-up analysis if no obvious blebs were observed or if significant hemorrhage occurred at the time of injection.

### Protein Extraction, SDS-PAGE, and Immunoblotting

To extract proteins from HEK293T/17 cells, cells in 6-well plates were briefly rinsed with ice-cold PBS and lysed with 300 μl of ice-cold lysis buffer (50 mM HEPES pH 7.0, 150 mM NaCl, 2 mM EGTA, 2 mM MgCl₂, 1% Triton X-100) supplemented with Protease Inhibitor Cocktail (Bimake). Lysates were centrifuged at 20,000×g for 15 minutes at 4° C. to precipitate insoluble materials.

To extract proteins from mouse retinas, mice were euthanized by CO₂ asphyxiation followed by cervical dislocation. Eyes were enucleated and anterior segments were removed using micro-dissecting scissors. Collected tissues were homogenized with a pestle in a lysis buffer (50 mM HEPES pH 7.0, 150 mM NaCl, 2 mM EGTA, 2 mM MgCl₂, 1% Triton X-100) supplemented with the Protease Inhibitor Cocktail. Homogenates were centrifuged at 20,000×g for 15 minutes at 4° C. to precipitate insoluble materials.

Supernatants were mixed with NuPAGE LDS Sample Buffer (Invitrogen) and Reducing Agent (Invitrogen), and 40-50 μg of proteins were loaded per lane on NuPAGE 4-12% (wt/vol) Bis-Tris gels (Invitrogen; for all genes except CDH23) or 3-8% Tris-Acetate gels (Invitrogen; for CDH23). Proteins were transferred onto nitrocellulose membranes (BioRad) and immunoblotting was performed following standard protocols. Proteins were detected by using horse radish peroxidase (HRP)-conjugated secondary antibodies and SuperSignal West Dura Extended Duration Substrate (Thermo Scientific). Images were taken with a ChemiDoc Imaging system (Bio-Rad).

### Immunohistochemistry

Mouse eyes were collected, fixed, and embedded in Neg-50 Frozen Section Medium (Richard-Allan Scientific) as described previously (61, 63). The frozen eye cups were mounted on a cryostat chuck and oriented to obtain sections along the superior-inferior axis. Retinal sections (8-μm thick) from the middle half of the eye cups were collected at intervals of every 5-6 sections using a CryoStar NX70 cryostat (Epredia) and used for immunohistochemistry. The immunostaining and imaging procedures were described previously (61).

### Electroretinography (ERG)

The ERG recording procedures were described previously (64). Scotopic and photopic ERG responses were measured with 15 flashes of stimulus light at 0.01 cd×s/m² and 3.0 cd×s/m², respectively (color temperature: 6500K). Statistical analyses were performed using two-tailed, two-sample t-tests with unequal variances. A p-value of less than 0.05 was considered statistically significant.

### Results

### Development of Novel Lox Site Variants for Sequence-Specific, Unidirectional Recombination

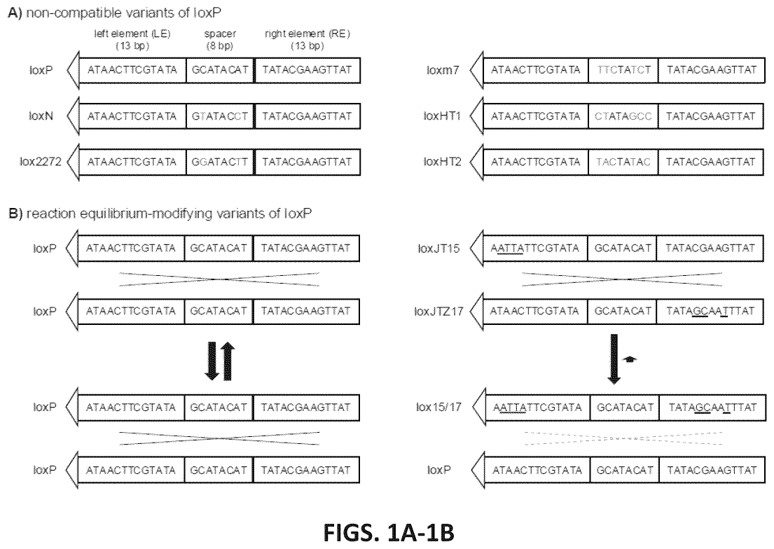

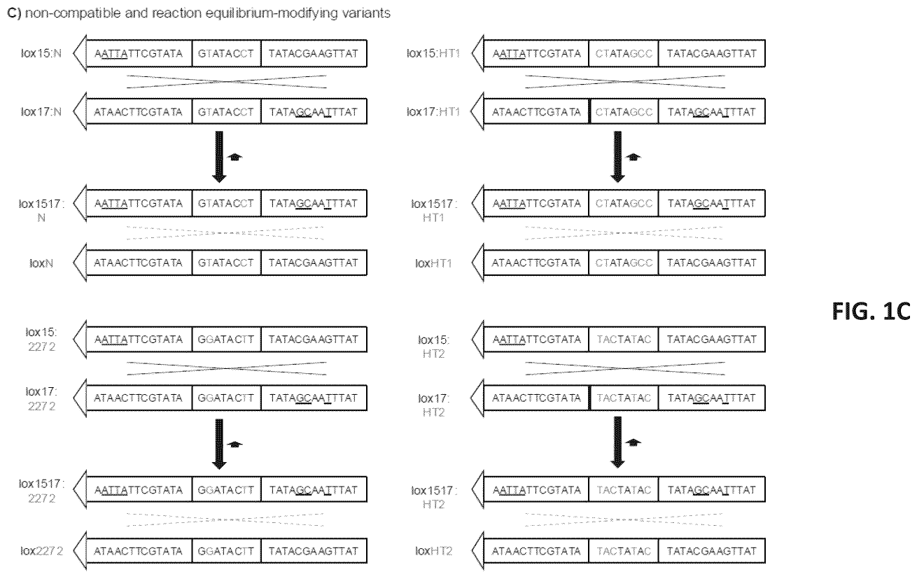

The canonical loxP site consists of two 13-bp inverted repeats (left and right elements; LE and RE, respectively) separated by an asymmetric 8-bp spacer/core sequence (FIG. 1A). While the left and right elements serve as the binding sites for the CRE recombinase, the spacer participates in the strand exchange reaction and determines the compatibility between lox sites (i.e., whether two lox sites can recombine or not) (33-35). The asymmetry of the spacer imparts directionality to the loxP site. There are two classes of lox site variants. One is non-compatible mutant variants, which include loxm7, loxN, and lox2272 (36-38) (FIG. 1A). These variants have mutations within the spacer sequence, and these mutations prevent strand exchange (and consequently recombination) between non-compatible lox sites while allowing recombination between homologous (or compatible) sites. A high-throughput screen identified fully non-compatible and promiscuous lox sites (39). The second group comprises reaction equilibrium-modifying variants (FIG. 1B). These variants have mutations in either LE or RE, but not in both (e.g., loxJT15, loxJTZ17, lox71, and lox66) (40, 41). Single-element mutations do not affect the binding of CRE to the lox site, and recombination between these mutant lox sites occurs as efficiently as between canonical loxP sites. However, recombination between LE and RE single mutants produces an LE/RE double mutant and a canonical loxP site. The presence of mutations in both LE and RE significantly reduces the affinity of the LE/RE double mutant to CRE, making it a poor substrate. While the recombination between canonical loxP sites is fully reversible as the initial substrates and the products have the same lox sites, the reaction equilibrium is drastically shifted toward the forward direction when LE and RE single mutants are used as substrates because the reverse reaction is much slower than the forward reaction. This causes the CRE-mediated recombination nearly unidirectional when the reaction equilibrium modifying lox sites are used.

Although the CRE-lox system is highly efficient, canonical loxP sites (or any single species of lox sites) cannot be used to combine more than two DNA molecules. When there are two or more loxP sites in a single DNA molecule, the intervening “floxed” sequence is rapidly excised (Figure S1A). The reverse reaction (i.e., insertion) is much slower than the forward reaction. Furthermore, if all DNA fragments have the same lox sites, recombination can occur in any combination (Figure S1B), preventing the specification of the order of DNA segments in end products, leading to the production of unintended, non-functional products. Lastly, since the CRE-lox recombination is fully reversible, the reconstituted DNA constantly goes through the assembly-disassembly cycle, limiting the yield of the reconstituted DNAs (Figure S1C). In principle, the yields of reconstituted products are 50%, 25%, and 12.5% when 2, 3, and 4 fragments are used as substrates, respectively, even when the excision and the order of DNA segment problems are disregarded.

To enable the reconstitution of large genes using multiple AAV vectors and the CRE-lox DNA recombination system, novel lox sites were devised by combining non-compatible and reaction equilibrium modifying lox site variants (FIG. 1C). The loxJT15-loxJTZ17 pair was chosen for the reaction equilibrium modifying mutants because this pair was the most effective in inhibiting reverse-direction recombination (40). For the non-compatible lox sites, loxN, lox2272, loxm7, and two additional lox sites identified by a high-throughput screen with spacer sequences CTATAGCC (named loxHT1 herein) and TACTATAC (loxHT2) were chosen (39). The loxN-based pair, for example, was generated by replacing the spacer sequence of loxJT15 and loxJTZ17 (GCATACAT) with that of loxN (GTATACCT). If these hybrid lox sites are fully non-compatible with one another, they will prevent the excision of intervening sequences and unintended recombinations (Figure S1D and E). At the same time, they will significantly increase the yield of reconstituted DNA products by inhibiting reverse reactions, particularly when using three or more AAV vectors (Figure S1F).

To assess the compatibility of the developed hybrid lox sites in mammalian cells, GFP expression cassettes capable of tracking recombination events between different lox sites were designed (FIG. 2 and Figure S2). The first reporter construct, loxP-2272, was designed to survey the compatibility of loxJT15 with four hybrid lox sites (FIG. 2A). The reporter is composed of a CMV promoter, a GFP coding sequence (CDS), a 156-bp segment from the human CEP290 C-terminus (C290C; amino acids 2428-2479; in frame with GFP), and a stop codon. A loxJT15 site (15:P) was inserted between GFP and C290C such that recombination events between loxJT15 and any downstream lox sites would result in the excision of C290C and the production of new GFP fusion proteins with an identification tag (Figure S2). FLAG, HA, MYC, and V5 tags were used to report the recombination of loxJT15 with loxJTZ17:m⁷ hybrid (hereafter denoted as 17:m⁷ for brevity), 17:HT1, 17:HT2, and 17:2272 sites, respectively. Recombination between loxJT15 (15:P) and 17:HT1, for instance, results in the production of GFP+HA fusion proteins (˜30 kDa). Of note, recombination events not involving the 15:P site, such as between 17:m⁷ and 17:2272, do not lead to the fusion of associated tags with GFP and therefore go unreported. Four additional reporter constructs (loxP-N, lox2272-N, loxP-HT2, and lox2272-HT2) were generated to examine the compatibility among the hybrid lox sites that we have developed (FIG. 2A).

When transfected into HEK293T cells, these reporters produced 35-kDa GFP+C290C fusion proteins, which could be detected by our CEP290 antibody (FIG. 2B and Figure S2). However, when cotransfected with a CRE expression plasmid (pAAV-EF1a-CRE), reporters containing the 17:N site (i.e., loxP-N and lox2272-N) expressed GFP+V5 fusion proteins. In contrast, no new GFP fusion proteins were detected in cells transfected with loxP-2272, loxP-HT2, and lox2272-HT2. These results suggest that the spacer of loxN is partially compatible with those of loxP and lox2272, while loxP and lox2272 are fully incompatible with each other and with loxm7, loxHT1, and loxHT2. Since these reporters are designed to disclose only recombination events that involve the first lox site, which is linked to GFP, the compatibility among downstream lox sites (i.e., loxm7, loxHT1, and loxHT2) was not tested in this assay. As a positive control for Western blotting (lane 12), lysates from cells transfected with FLAG-LZTFL1, HA-LZTFL1, and MYC-BBS1 expression vectors were included to rule out the possibility of Western blotting failure, with b-actin serving as a loading control. Based on these results, the spacer sequences of loxP, lox2272, and loxHT1 were selected for the assembly of up to four AAV vector genomes. The spacers of loxm7 and loxHT2 may be used as alternatives to loxHT1 in this assembly strategy.

### CRE-Lox-Mediated Reconstitution of Large Genes Delivered by Tripartite AAV Vectors

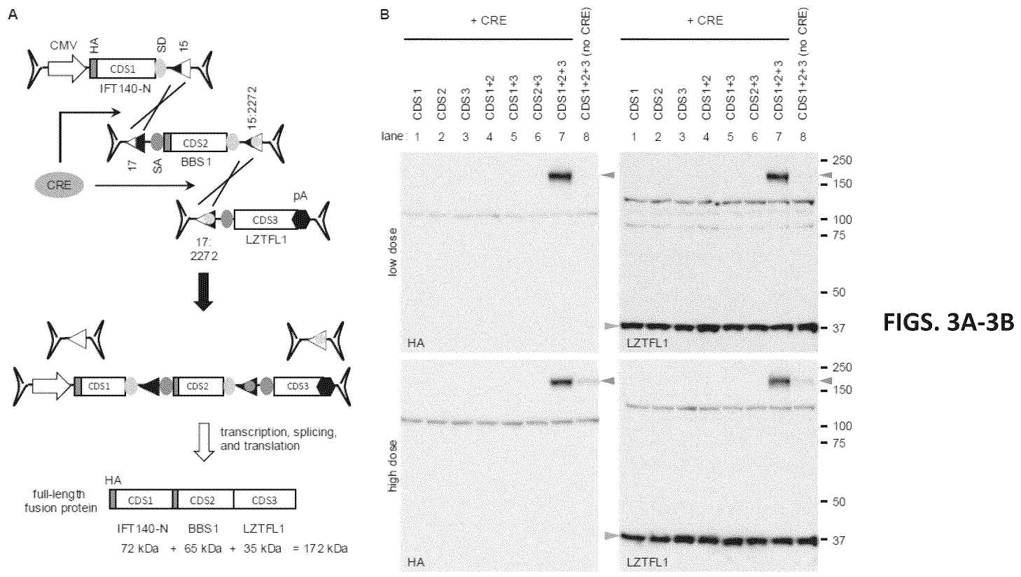

To evaluate the feasibility of the approach in delivering large genes using tripartite AAV vectors, a set of three AAV vectors were designed containing the coding sequences of three human genes: 5′ 1,923 bp of IFT140 (IFT140-N), full-length BBS7, and full-length LZTFL1 (FIG. 3A). The first vector comprises a CMV promoter, 5′ 1,923 bp of the IFT140 CDS, a splice donor (SD) site, and a loxJT15 site. An HA tag sequence was added to the 5′ end of IFT140 to facilitate the detection of expressed proteins. The second vector contains a loxJTZ17 site, a splice acceptor (SA) site, the BBS1 CDS (1,775 bp; including an HA tag and two linker sequences), an SD site, and a lox15:2272 site. The third vector is composed of a lox17:2272 site, a SA site, the LZTFL1 CDS (988 bp; including a linker sequence), and a bovine growth hormone (BGH) transcription termination signal. Recombination between these three AAV vector genomes in the correct arrangement will lead to the reconstitution of an expression cassette encoding IFT140N+BBS1+LZTFL1 fusion proteins.

These AAV vectors, all utilizing serotype 2, were transduced individually or in various combinations to HEK293T cells at two different doses. The “low” dose involved transduction at a multiplicity of infection (MOI) of 1.5×10⁴ genome copies (GC) per cell of each vector, while the “high” dose utilized an MOI of 6.0×10⁴ GC/cell of each vector. CRE recombinase was delivered via a separate AAV vector (AAV2/2-EF1a-CRE) with an MOI of 0.3×10⁴ GC/cell for the low dose and 1.2×10+GC/cell for the high dose. As shown in FIG. 3B, robust expression of the IFT140N+BBS1+LZTFL1 fusion protein (red arrowheads) was observed using both HA and LZTFL1 antibodies at both low and high doses (upper and lower panels, respectively). The migration rate of the protein was consistent with the predicted molecular weight of the full-length fusion protein, approximately 172 kDa. Notably, no protein production was detected when the three AAV vectors were transduced individually or any of the three was omitted. Although the expression of IFT140N+BBS1+LZTFL1 fusion proteins was not detected in the absence of CRE (lane 8) at the low dose, a small amount was detectable at the high dose (averaging 7% of CRE co-transduced cells; n=3). These proteins are likely attributed to spontaneous and fortuitous recombination of the three AAV vector genomes in the correct configuration. Endogenous LZTFL1, marked by blue arrowheads, served as a loading control. These data indicate that the CRE- and hybrid lox site-mediated reconstitution is significantly more efficient than the trans-splicing approach for reconstituting large genes.

### CRE-Lox-Mediated Reconstitution of Large Genes Delivered by Quadripartite AAV Vectors

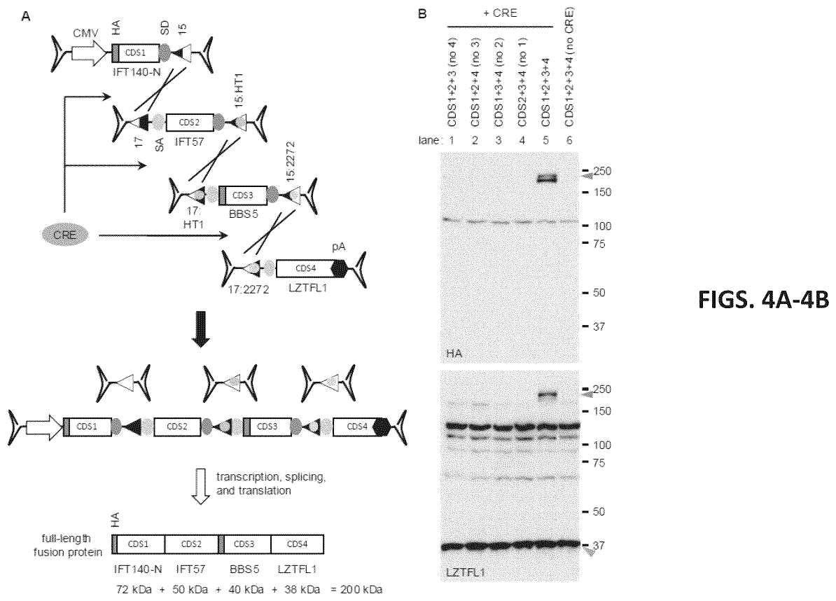

The approach was expanded to utilize quadripartite AAV vectors. For a proof-of-concept demonstration, AAV vectors were designed containing the CDSs of the 5′ 1,923 bp of IFT140, IFT57, BBS5, and LZTFL1 (FIG. 4A). The first and fourth AAV vectors that contained the IFT140-N and LZTFL1 CDSs were the same ones used in the tripartite set above. The second vector was constructed with a loxJTZ17 site, a SA site, the IFT57 CDS (1,330 bp; including two linker sequences), a SD site, and a lox15:HT1 site. The third vector was composed of a lox17:HT1 site, a SA site, the BBS5 CDS (1,090 bp; including an HA-tag and two linker sequences), a SD site, and a lox15:2272 site. If recombination occurs as intended, it will result in the reconstitution of an expression cassette encoding IFT140N+IFT57+BBS5+LZTFL1 fusion proteins with a predicted molecular weight of ˜200 kDa.

These AAV vectors were transduced into 293T cells in various combinations at an MOI of 2.5×10⁴ GC/cell of each vector. The AAV-EF1a-CRE vector was transduced at an MOI of 0.5×10⁴ GC/cell. Upon transduction of the quadripartite AAV vectors and AAV-EF1a-CRE, the production of IFT140N+IFT57+BBS5+LZTFL1 fusion proteins was readily detected by immunoblotting using HA and LZTFL1 antibodies (FIG. 4B; lane 5). The fusion protein appeared to be unstable, with some instances of truncation near the C-terminus, resulting in doublets. When any of the four AAV vectors was omitted, the full-length fusion protein was not produced (lanes 1-4). Furthermore, in the absence of CRE, the fusion protein was not detected (lane 6). These data validate the successful reconstitution of a gene expression cassette delivered by quadripartite AAV vectors and underscore the efficacy of our CRE-lox-mediated recombination approach.

### CRE-Lox-Mediated Reconstitution of IFT140 Delivered by Bipartite AAV Vectors

The CRE-lox-mediated DNA recombination approach was applied to IFT140, a gene associated with retinitis pigmentosa (RP) and short-rib thoracic dysplasia (42-44). Although the full-length CDS of IFT140 (4,389 bp) is small enough to be accommodated within a single AAV vector, additional regulatory sequences such as a promoter, a transcription termination signal, and two inverted terminal repeats (ITRs) must be included in the gene therapy vector, and the addition of such sequences makes the IFT140 expression cassette to exceed the AAV's packaging capacity. Therefore, at least two AAV vectors are required to deliver the IFT140 gene. Dual AAV-IFT140 vectors were constructed using the CRE-lox-mediated DNA recombination approach and examined whether full-length IFT140 could be successfully delivered by this method.

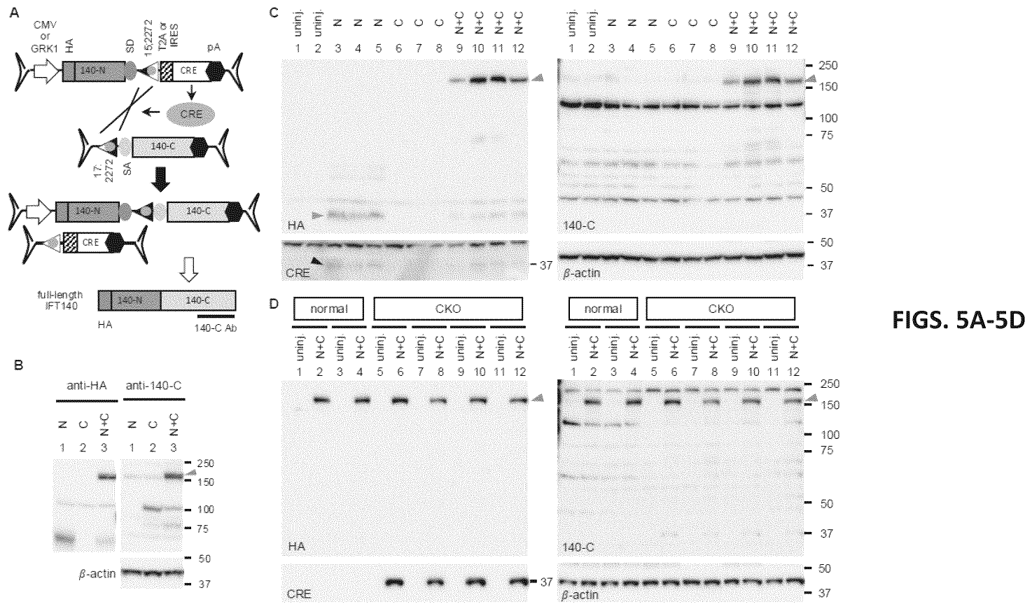

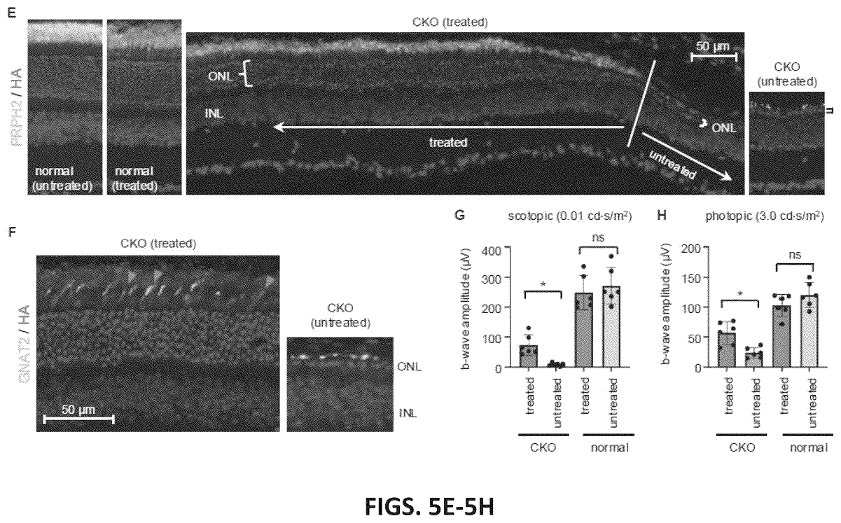

The 5′ vector was composed of a CMV promoter, 5′ 1,923 bp of the IFT140 CDS, an SD site, and a lox15:2272 site, and the 3′ vector contained a lox17:2272 site, an SA site, the rest of the IFT140 CDS (2,466 bp), and a BGH transcription termination signal (FIG. 5A). Since the combined payload capacity of two AAV vectors is ˜9 kb and the size of the IFT140 expression cassette is 6-6.5 kb, there is space to accommodate the CRE gene within the dual AAV-IFT140 vectors. We incorporated the CRE CDS, along with an N-terminal T2A “self-cleaving” peptide, after the lox15:2272 site in the 5′ vector. A BGH polyA signal was also added following the CRE gene. The inclusion of CRE in this configuration not only eliminates the need for a separate AAV vector for CRE delivery but also offers an additional benefit of CRE “self-inactivation” by causing the separation of the CRE CDS from its promoter as recombination progresses. To facilitate protein detection, an HA tag was introduced at the N-terminus of IFT140. Additionally, an IFT140 antibody (140-C Ab), which was raised against human IFT140 aa1114-1462, was used to detect the C-terminal portion of the protein.

Dual AAV-CMV-IFT140 vectors (with serotype 2) were transduced into 293T cells at an MOI of 3×10⁴ GC/cell for each vector. Upon transduction, the 5′ vector produced a ˜70 kDa protein detectable by the HA antibody (FIG. 5B; lane 1). The 3′ vector produced a ˜100 kDa protein (lane 2), which is presumably due to the intrinsic promoter activity of the ITRs (45). When both the 5′ and 3′ vectors were co-transduced, robust expression of full-length IFT140 was observed (lane 3; red arrowhead), demonstrating efficient reconstitution of the IFT140 expression cassette in 293T cells.

### Dual AAV-IFT140 CRE-Lox Vectors Prevent or Delay Retinal Degeneration in a Mouse Model of IFT140-Associated Retinitis Pigmentosa

Whether the CRE/lox-based dual AAV-IFT140 vectors could produce full-length IFT140 proteins in vivo and prevent retinal degeneration caused by the loss of IFT140 function was investigated. Since IFT140 is a relatively low-abundance protein, the rhodopsin kinase (GRK1) promoter was used, which is active in both rods and cones (46), instead of the CMV promoter (FIG. 5A). Additionally, to minimize the potential impact of producing N-terminal truncated proteins, the size of the IFT140-N fragment were minimized to 810 bp and the remaining 3,576 bp was placed in the 3′ vector, which lacked a promoter. These AAV vectors were prepared with the Anc80L65 serotype (4) and delivered to mouse retinas via subretinal injections.

The dual AAV-GRK1p-IFT140 vectors were first administered into the eyes of 1- to 2-month-old wild-type mice and examined IFT140 protein production by immunoblotting (FIG. 5C). To this end, animals received the dual AAV-GRK1p-IFT140 vectors either individually (n=3) or in combination (n=4) at a dose of 8×10⁸ GC of each vector. Treated eyes were collected two weeks post-injection, and protein extracts were analyzed by SDS-PAGE followed by immunoblotting. When injected individually, the S′ vector produced ˜37 kDa truncated protein products (lanes 3-5; blue arrowhead), while no protein production was observed with the 3′ vector (lanes 6-8). However, when the 5′ and 3′ vectors were co-delivered, full-length IFT140 proteins were readily detected with both HA and IFT140-C antibodies in all four injected animals (lanes 9-12). These data underscore the reliability of the CRE/lox-mediated DNA reconstitution approach for delivering large genes using AAV.

The therapeutic efficacy of the dual AAV-GRK1p-IFT140 vectors in Ift140 mutant mice was then evaluated. Because constitutive inactivation of Ift140 is embryonic lethal in mice, Ift140 conditional knockout (CKO) mice were used, in which exon 7 is floxed (47, 48). For retina-specific Ift140 ablation, the iCre75 driver (also known as rhodopsin-iCRE; (49)) was used to selectively ablate Ift140 expression in rod photoreceptors. Of note, iCre75-driven CRE activity is detectable as early as post-natal day (P) 7 (49). It also should be noted that the incompatibility between loxP (in the Ift140 floxed allele) and lox2272 (in the AAV-IFT140 vectors) sites prevents recombination between the Ift140fl alleles and the AAV vector genomes. Since retinal degeneration is already noticeable at P24 and progresses rapidly in Ift140^{fl/fl};iCre75⁺ mice (Figure S3), the AAV gene therapy vectors were delivered at an early stage of degeneration (P16).

To confirm the production of full-length IFT140 proteins in Ift140 CKO (Ift1402^{fl/fl};iCre75⁺) mice, treated and untreated contralateral eyes were collected at P55 (39 days post-injection), and protein extracts were subjected to SDS-PAGE followed by immunoblotting. As shown in FIG. 5D, full-length IFT140 expression was observed in all CKO mice injected (n=4). Notably, the 37-kDa IFT140 N-terminal truncated protein product was no longer detected in either CKO or normal (Ift140^{fl/fl};iCre75⁻) mouse eyes, suggesting that unassembled 5′ vectors may have been depleted by this time. Consistent with this, CRE expression was not detected in treated normal mouse eyes. The CRE proteins present in injected CKO mouse eyes are likely (or mostly) derived from the iCre75 transgene rather than the 5′ AAV-IFT140 vector. The absence of CRE in uninjected eyes is due to the complete loss of rod photoreceptors in CKO mice by this age (see below).

To assess whether the dual AAV-GRK1p-IFT140 vectors could prevent retinal degeneration, retinal sections were examined from treated and untreated contralateral eyes of normal (n=2) and Ift140 CKO (n=3) mice using immunohistochemistry at P52 (FIGS. 5E and F). HA, PRPH2, and GNAT2 antibodies were used to visualize IFT140, photoreceptor outer segments, and cone outer segments, respectively. In untreated CKO mouse eyes, the vast majority of photoreceptor cells had been lost by this age, with only 1-2 rows of photoreceptor cell nuclei remaining. In contrast, 5-8 rows of photoreceptor nuclei were preserved within the central two-thirds of the treated regions (n=3 mice). HA-IFT140 expression was also observed in cones (FIG. 5F; red arrowheads). In normal mice, the administration of AAV-GRK1p-IFT140 vectors did not appear to cause obvious toxic effects, at least at the dose used (8×10⁸ GC of each vector).

Lastly, the light responsiveness of the treated eyes was evaluated by electroretinography (ERG) at P45. To assess the rod function, mice were dark-adapted and subjected to dim light flashes (0.01 cd×s/m²) (FIG. 5G). In Ift140 CKO mice, untreated contralateral eyes exhibited minimal ERG responses, with an average b-wave amplitude±standard deviation (SD) of 8.7±5.2 μV (n=6). In contrast, significant ERG responses were detected in treated eyes, with an average amplitude of 73.2±34.3 μV (n=6). Statistical analysis using a two-tailed Student's t-test revealed a significant difference between the two groups (p=0.005). No significant differences were observed between treated and untreated normal eyes (248.2±57.2 μV vs. 271.4±61.7 μV; n=6; p=0.516). Cone function was evaluated by stimulating eyes with bright light flashes (3.0 cd·s/m²) after light adaptation (FIG. 5H). Although iCre75 was only expressed in rods, cone function was also reduced in Ift140 CKO mice at P45. However, eyes treated with the AAV-GRK1p-IFT140 gene therapy vectors exhibited significantly higher ERG responses, with an average b-wave amplitude of 57.0±19.1 μV, (n=6) compared to untreated contralateral eyes (23.8±8.6 μV, n=6; Student's t-test p=0.006). Similar to the scotopic ERG results, no significant differences were observed in photopic ERG between treated and untreated eyes (102.9±18.3 μV vs. 112.1±30.7 μV, n=6, p=0.549) in normal mice. A longer-term study is currently underway. Overall, these findings indicate that the dual AAV-GRK1p-IFT140 vectors can effectively prevent or delay retinal degeneration in Ift140 CKO mice.

### Reconstitution of PCDH15 by CRE-Lox-Mediated Recombination

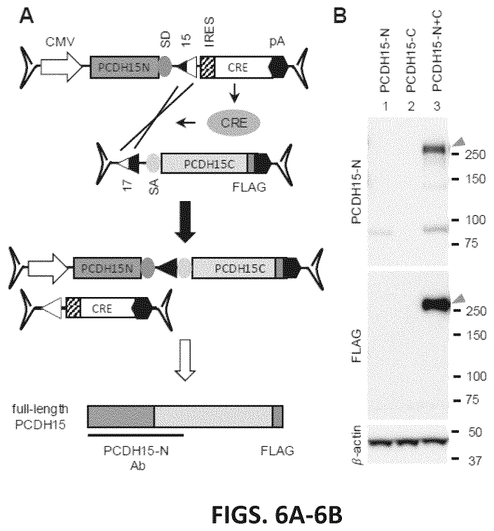

Mutations in PCDH15 cause Usher syndrome type 1F (USH1F), which is characterized by profound congenital hearing impairment and progressive vision loss (50-52). The full-length human PCDH15 CD spans 5,865 bp, necessitating two AAV vectors for delivery. The CRE-lox approach was applied to PCDH1 and created dual AAV vectors (FIG. 6).

The full-length human PCDH15 CDS was divided into two segments (1,932 bp and 3,933 bp) and inserted into the 5′ and 3′ vectors, respectively (FIG. 6A). Similar to the IFT140 gene, the CRE CDS was included within the 5′ vector, but in this case, an internal ribosome entry site (IRES) was used instead of the T2A self-cleaving peptide. Additionally, we introduced a FLAG tag to the C-terminus of PCDH15 to facilitate protein detection. For the detection of the N-terminal portion of PCDH15, a polyclonal antibody (PCDH15-N Ab) raised against recombinant human PCDH15 protein (aa Q27-A1376) was used.

When transduced to HEK293T cells (at an MOI of 3×10⁴ GC/cell for each vector), the 5′ vector produced an ˜85 kDa protein detected by the PCDH15-N antibody, while no protein production was observed from the 3′ vector (FIG. 6B, lanes 1 and 2). However, when both vectors were transduced together, robust expression of full-length PCDH15 was observed (lane 3; red arrowheads). These results demonstrate that the CRE-lox-based dual AAV-PCDH15 vectors are an effective method for delivering full-length PCDH15 gene.

### Reconstitution of CEP290 by CRE-Lox-Mediated Recombination

CEP290 mutations are associated with various ciliopathies, ranging from isolated retinal dystrophy to syndromic conditions such as Bardet-Biedl syndrome, Joubert syndrome, and Meckel-Gruber syndrome (7-9, 53-55). Although the full-length human CEP290 CDS (7,440 bp) can be accommodated in two AAV vectors, a better outcome was obtained when CEP290 was split into three AAV vectors in a prior study to develop CEP290 gene therapy vectors using the split intein strategy (30). However, even with the optimized set, the yield of full-length CEP290 was very low, and a large amount of truncated protein products remained unspliced.

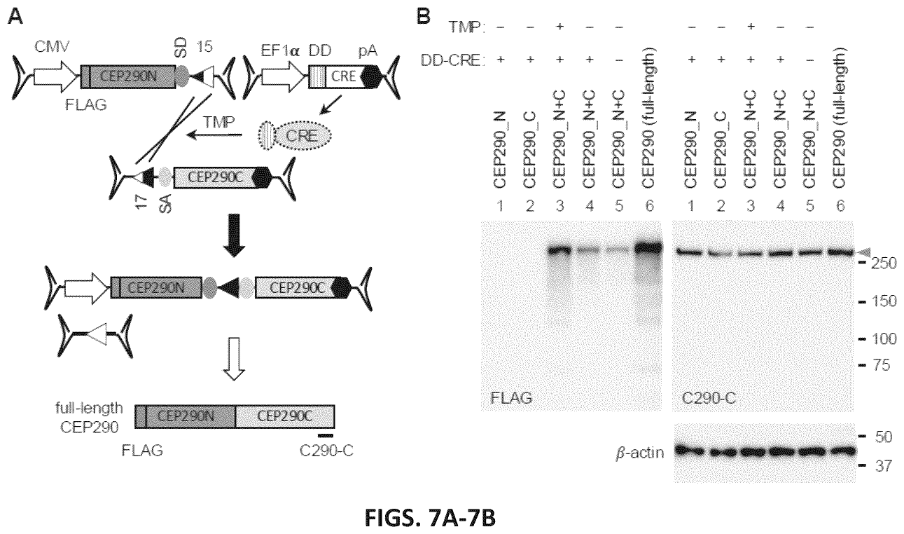

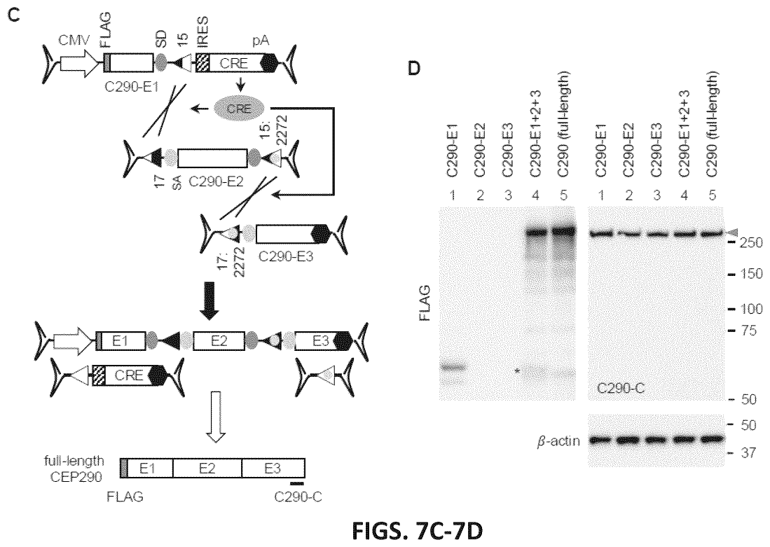

We applied the CRE-lox approach to CEP290 and developed bipartite and tripartite AAV-CEP290 vectors. For the bipartite set (FIG. 7A), the CEP290 CDS was divided into two fragments (3,527 bp and 3,913 bp) and inserted into the 5′ and 3′ vectors, respectively. CRE was delivered through a separate AAV vector (AAV-EF1a-DD-CRE). To prevent constitutive overexpression of CRE, the dihydrofolate reductase destabilizing domain (DD)-fused CRE was adopted (56, 57), which undergoes rapid degradation through the proteasomal pathway but can be temporarily stabilized by the addition of trimethoprim (TMP) (Figure S3). For the tripartite set (FIG. 7C), CEP290 was split into 3 segments (5′ (E1): 1,065 bp, middle (E2): 2,913 bp, and 3′ (E3): 3,462 bp), and the CRE gene was included within the 5′ vector. The loxJT15 and loxJTZ17 pair was used to join the 5′ and middle vectors and the lox15:2272 and lox17:2272 pair was used for the middle and 3′ vectors. A FLAG tag sequence was introduced at the 5′ end of CEP290 to facilitate the detection of the N-terminal portion of CEP290 in both sets, and the CEP290-C antibody was used to detect the C-terminal portion of CEP290.

To evaluate the reconstitution and expression of CEP290 from the bipartite AAV-CEP290 vectors, HEK293T cells were transduced with the dual AAV-CEP290 vectors at an MOI of 3×10⁴ GC/cell of each vector and AAV-EF1a-DD-CRE at an MOI of 1×10⁴ GC/cell. After transduction, cells were treated with 10 μM TMP for 48 hours to stabilize DD-CRE, and CEP290 expression was assessed by immunoblotting (FIG. 7B). FLAG-tagged, full-length CEP290 expression was detectable in cells transduced with all three AAV vectors and treated with TMP (lane 3). FLAG-CEP290 expression was also detectable without TMP treatment (lane 4), but at a significantly lower level (average 24% of TMP-treated cells; n=3). A small amount of FLAG-CEP290 was observed in the absence of CRE (lane 5; average 9% of TMP-treated cells; n=3), representing CEP290 gene reconstitution via spontaneous recombination between the dual AAVCEP290 vectors. Plasmid DNAs encoding full-length CEP290 were transfected and used as a positive control (lane 6). Over-expressed CEP290 appears to be unstable and degraded, and consequently, one could not observe increased CEP290 protein levels beyond endogenous expression using the CEP290-C antibody. b-actin was used as a loading control.

The tripartite AAV-CEP290 vectors were transduced into 293T cells at an MOI of 3×10⁴ GC/cell of each vector, and FLAG-CEP290 expression was examined as above. As shown in FIG. 7D, full-length CEP290 expression was detectable only when all three AAV vectors were transduced. These data demonstrate that both bipartite (with a separate AAV-CRE vector) and tripartite AAV-CEP290 vectors efficiently deliver the full-length CEP290 gene to target cells. Reconstitution of CDH23 by CRE-lox mediated recombination

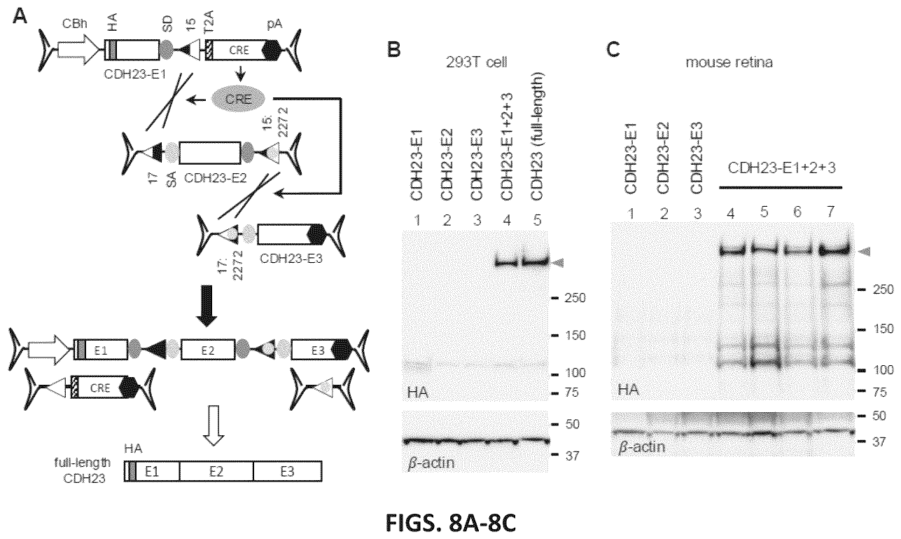

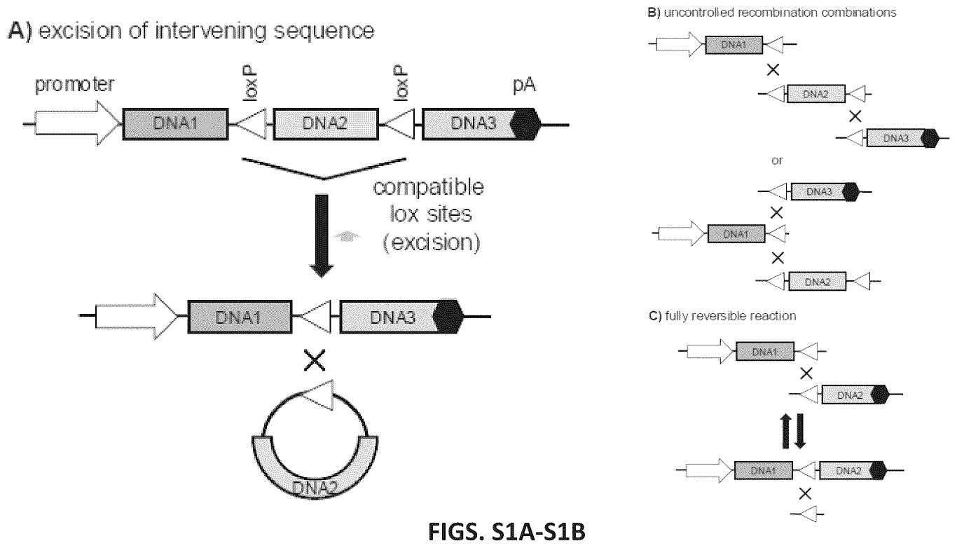

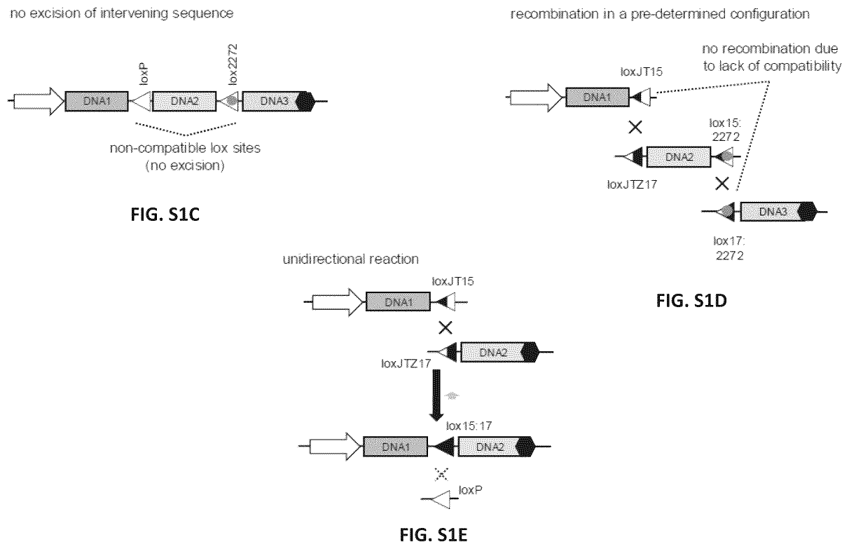

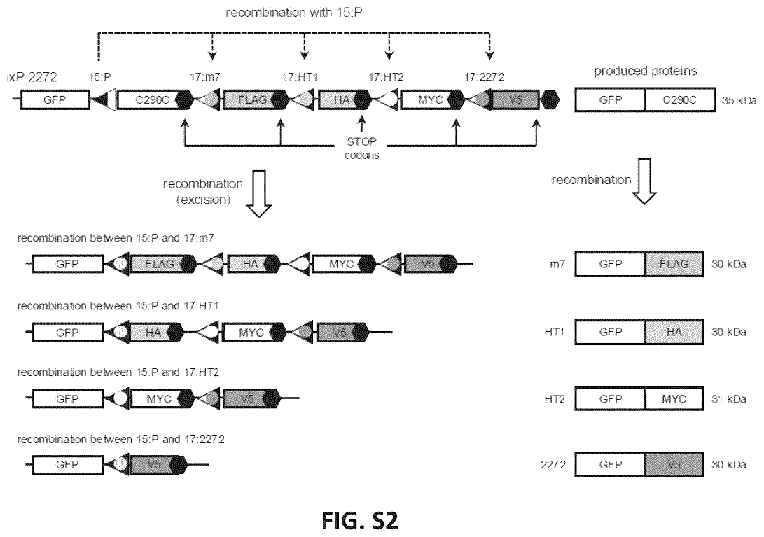

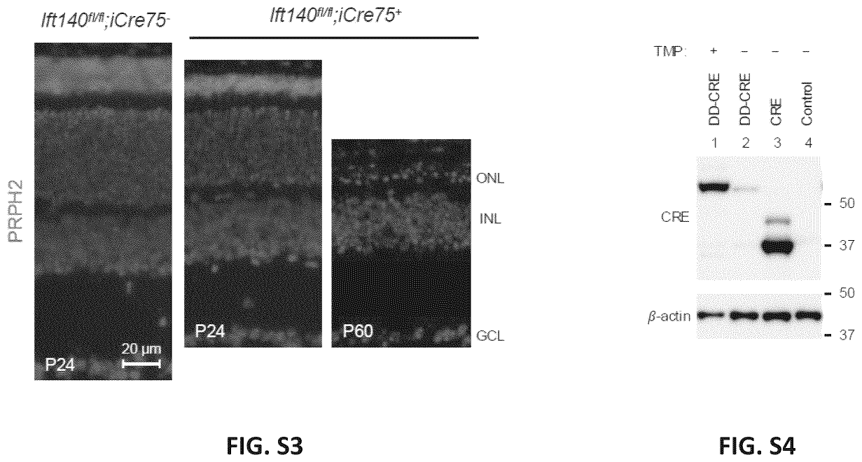

The CRE-lox-mediated DNA recombination approach was then applied to CDH23 gene therapy vectors. Inactivating mutations of CDH23 cause Usher syndrome type ID (USHID) (13, 14). The full-length human CDH23 CDS spans 10,065 bp, requiring three AAV vectors for delivery. Since the total payload capacity of three AAV vectors is ˜14 kb (excluding ITRs), the CDH23 CDS was split into 3 fragments (5′ vector (E1): 2,176 bp, middle vector (E2): 4,077 bp, and 3′ vector (E3): 3,812 bp) and included the CRE gene (with a T2A peptide) in the 5′ vector (FIG. 8A). The loxJT15 and loxJTZ17 pair was used to join the 5′ and the middle vectors and the lox15:2272 and lox17:2272 pair was used for the middle and the 3′ vectors. CDH23 is a type-I single transmembrane protein with an N-terminal signal peptide, and an HA tag was inserted after the signal peptide for protein detection. A CBh promoter and a BGH poly A signal were used as a promoter and a transcription termination signal, respectively.

To test whether the tripartite AAV-CDH23 vectors could deliver full-length CDH23, HEK293T cells were transformed with these vectors at an MOI of 3×10⁴ GC/cell of each vector (serotype AAV2) and examined the production of full-length CDH23 proteins by immunoblotting. When all three vectors were co-transduced, robust expression of CDH23 was observed (FIG. 8B; red arrowhead). A plasmid containing a full-length CDH23 expression cassette was used as a positive control (lane 5), and b-actin was used as a loading control. To test whether these vectors could be used to deliver CDH23 in vivo, the tripartite AAV-CDH23 vectors were administered to wild-type mouse eyes by subretinal injections (serotype: AAV5, dose: 3×10⁹ GC per vector). Consistent with the results in 293T cells, full-length CDH23 proteins were readily detected in all eyes injected with the tripartite AAV vectors (FIG. 8C, lanes 4-7).

### Discussion

Reconstitution of therapeutic genes via the CRE-lox-mediated DNA recombination offers several advantages compared to other approaches that have been reported thus far.

First, compared to spontaneous recombination-dependent approaches such as trans-splicing, overlapping, and hybrid dual or triple AAV approaches (17-20), the CRE-lox-mediated recombination drastically increases the recombination efficiency and the yield of correctly reconstituted genes. This is especially true when tripartite or quadripartite AAV vectors are used. The use of non-compatible, hybrid lox sites prevent the excision of floxed sequences, ensures recombination in a pre-determined configuration, and inhibits the disassembly of reconstituted genes (Figure S1D-F). The enhanced efficiency and yield reduce the number of AAV particles needed to transduce target cells, and the use of fewer AAV vectors reduces the potential risks of viral vector-derived toxicity and inflammation.

Second, the CRE-lox-mediated DNA recombination approach provides more flexibility regarding splitting positions compared to the protein trans-splicing approach. The efficiency of protein trans-splicing is influenced by the amino acid residues adjacent to split inteins (25, 31, 32). The first residue within the Cextein is particularly important, and Cys, Ser, and Thr residues are strongly preferred. This constraint limits the number of possible locations where a protein may be split. Moreover, protein truncations can affect a protein's structure, stability, and localization, and these factors also influence the overall efficiency and yield of the protein reconstitution. If the target gene encodes a transmembrane or secreted protein (e.g., PCDH15 and CDH23), the topology and secretion of each protein fragment should be considered when determining splitting positions. Ideal splitting positions for the split intein approach should support the protein trans-splicing process and have minimal or no effect on the folding and stability of each truncated protein fragment and reconstituted proteins. Additionally, the truncated protein products should be localized to the same compartment or close locations to increase the likelihood of engagement, while the size of each fragment should be small enough to fit into single AAV vectors. Therefore, identifying optimal splitting positions could be challenging and usually involves comparing multiple candidate sites empirically. And the complexity increases if the target gene requires 3 or 4 AAV vectors. In contrast, when using the CRElox approach, protein structure, stability, localization, and topology are not factors to consider since the reconstitution occurs at the DNA level. Cargo capacity expansion can be achieved by simply adding additional sets of non-compatible lox sites to AAV vectors.

Another significant advantage of the CRE-lox approach over protein trans-splicing is the lack or minimal production of truncated proteins. Protein trans-splicing requires the production of “half” proteins before reconstitution, which can have dominant negative or harmful effects if continuously expressed. In contrast, truncated protein production is either absent or low when the CRE-lox strategy is used because the AAV vectors lack a promoter or a polyA signal, which stabilizes mRNA. Although ITRs have some intrinsic promoter activities (45), they are weak in most cells. When the CRE gene is included in the 5′ vector, a truncated protein and CRE are initially produced. However, as recombination progresses and the 5′ vector is converted to a full-length therapeutic cassette, the production of truncated proteins and CRE diminishes. The lack or minimal production of truncated proteins may be crucial for certain genes if such protein products are toxic to cells, and the “self-inactivation” feature of the CRE-containing 5′ vector provides an additional layer of safety to the CRE-lox approach.

Lastly, although the CRE-lox approach requires the delivery of CRE in addition to therapeutic genes, the practical payload capacity of AAV vectors with the CRE-lox approach is either comparable with or larger than that of the split intein-based approach. The protein trans-splicing approach requires each AAV vector to have its own promoter and transcription termination signal to produce therapeutic gene products and split inteins. The repeated inclusion of transcriptional regulatory elements erodes the AAV vector's combined payload capacity. In contrast, the CRE-lox approach only requires one promoter and one transcriptional termination signal for the entire vector set, which becomes more beneficial as more AAV vectors are needed.

One notable concern of the CRE-lox approach is the prolonged expression of CRE recombinase in transduced cells. Prolonged expression of CRE could lead to unintended recombination in the human genome, potentially resulting in unwanted mutations or genomic instability. Moreover, prolonged CRE expression could also lead to immune responses, which may limit the effectiveness of AAV gene therapies. This could potentially result in the destruction of cells expressing the therapeutic gene or a reduction in the efficacy of the AAV gene therapy over time. In this regard, the inclusion of CRE in 5′ vectors and being “self-inactivated” by recombination significantly reduces these risks. Alternatively, an inducible promoter or destabilizing domain fused CRE may be used to control CRE expression. Other site-specific DNA recombinases that display higher specificity than CRE may be used as well. It is noteworthy that while numerous transgenic rodent lines expressing CRE have been created and some phenotypes have been reported (58), constitutive expression of CRE does not appear to cause serious health concerns in rodents. However, a thorough investigation of genes or sites in the human genome that could be modified by CRE will be necessary. As with any medical treatment, it is crucial to carefully consider the potential risks and benefits associated with CRE-lox-dependent AAV gene therapies.

In summary, the CRE-lox approach described herein offers a simple, versatile, and efficient platform for producing AAV-based, generic gene replacement therapy vectors capable of delivering large genes. As this approach delivers full-length genes, the gene therapy vectors developed using this method are generally applicable to all patients with loss-of-function mutations.

### BIBLIOGRAPHY

1 Bennett, J. (2003) Immune response following intraocular delivery of recombinant viral vectors. Gene Ther, 10, 977-982.
2 Lugin, M. L., Lee, R. T. and Kwon, Y. J. (2020) Synthetically Engineered Adeno-Associated Virus for Efficient, Safe, and Versatile Gene Therapy Applications. ACS Nano, 14, 14262-14283.
3 Asokan, A., Schaffer, D. V. and Samulski, R. J. (2012) The AAV vector toolkit: poised at the clinical crossroads. Mol Ther, 20, 699-708.
4 Zinn, E., Pacouret, S., Khaychuk, V., Turunen, H. T., Carvalho, L. S., Andres-Mateos, E., Shah, S., Shelke, R., Maurer, A. C., Plovie, E. et al. (2015) In Silico Reconstruction of the Viral Evolutionary Lineage Yields a Potent Gene Therapy Vector. Cell Rep, 12, 1056-1068.
5 Wu, Z., Yang, H. and Colosi, P. (2010) Effect of genome size on AAV vector packaging. Mol Ther, 18, 80-86.
6 Stone, E. M., Andorf, J. L., Whitmore, S. S., DeLuca, A. P., Giacalone, J. C., Streb, L. M., Braun, T. A., Mullins, R. F., Scheetz, T. E., Sheffield, V. C. et al. (2017) Clinically Focused Molecular Investigation of 1000 Consecutive Families with Inherited Retinal Disease. Ophthalmology, 124, 1314-1331.
7 den Hollander, A. I., Koenekoop, R. K., Yzer, S., Lopez, I., Arends, M. L., Voesenek, K. E., Zonneveld, M. N., Strom, T. M., Meitinger, T., Brunner, H. G. et al. (2006) Mutations in the CEP290 (NPHP6) gene are a frequent cause of Leber congenital amaurosis. American journal of human genetics, 79, 556-561.
8 Perrault, I., Delphin, N., Hanein, S., Gerber, S., Dufier, J. L., Roche, O., Defoort-Dhellemmes, S., Dollfus, H., Fazzi, E., Munnich, A. et al. (2007) Spectrum of NPHP6/CEP290 mutations in Leber congenital amaurosis and delineation of the associated phenotype. Hum Mutat, 28, 416.
9 Baala, L., Audollent, S., Martinovic, J., Ozilou, C., Babron, M. C., Sivanandamoorthy, S., Saunier, S., Salomon, R., Gonzales, M., Rattenberry, E. et al. (2007) Pleiotropic effects of CEP290 (NPHP6) mutations extend to Meckel syndrome. Am J Hum Genet, 81, 170-179.
10 Valente, E. M., Silhavy, J. L., Brancati, F., Barrano, G., Krishnaswami, S. R., Castori, M., Lancaster, M. A., Boltshauser, E., Boccone, L., Al-Gazali, L. et al. (2006) Mutations in CEP290, which encodes a centrosomal protein, cause pleiotropic forms of Joubert syndrome. Nat Genet, 38, 623-625.
11 Sayer, J. A., Otto, E. A., O'Toole, J. F., Nurnberg, G., Kennedy, M. A., Becker, C., Hennies, H. C., Helou, J., Attanasio, M., Fausett, B. V. et al. (2006) The centrosomal protein nephrocystin-6 is mutated in Joubert syndrome and activates transcription factor ATF4. Nat Genet, 38, 674-681.
12 Coppieters, F., Lefever, S., Leroy, B. P. and De Baere, E. (2010) CEP290, a gene with many faces: mutation overview and presentation of CEP290base. Hum Mutat, 31, 1097-1108.
13 Bolz, H., von Brederlow, B., Ramirez, A., Bryda, E. C., Kutsche, K., Nothwang, H. G., Seeliger, M., del, C. S. C. M., Vila, M. C., Molina, O. P. et al. (2001) Mutation of CDH23, encoding a new member of the cadherin gene family, causes Usher syndrome type ID. Nat Genet, 27, 108-112.
14 Di Palma, F., Holme, R. H., Bryda, E. C., Belyantseva, I. A., Pellegrino, R., Kachar, B., Steel, K. P. and Noben-Trauth, K. (2001) Mutations in Cdh23, encoding a new type of cadherin, cause stereocilia disorganization in waltzer, the mouse model for Usher syndrome type ID. Nat Genet, 27, 103-107.
15 Trapani, I., Puppo, A. and Auricchio, A. (2014) Vector platforms for gene therapy of inherited retinopathies. Progress in retinal and eye research, 43, 108-128.
16 Trapani, I., Tornabene, P. and Auricchio, A. (2021) Large gene delivery to the retina with AAV vectors: are we there yet? Gene Ther, 28, 220-222.
17 Ghosh, A., Yue, Y., Lai, Y. and Duan, D. (2008) A hybrid vector system expands adeno-associated viral vector packaging capacity in a transgene-independent manner. Mol Ther, 16, 124-130.
18 Dyka, F. M., Boye, S. L., Chiodo, V. A., Hauswirth, W. W. and Boye, S. E. (2014) Dual adenoassociated virus vectors result in efficient in vitro and in vivo expression of an oversized gene, MYO7A. Hum Gene Ther Methods, 25, 166-177.
19 Carvalho, L. S., Turunen, H. T., Wassmer, S. J., Luna-Velez, M. V., Xiao, R., Bennett, J. and Vandenberghe, L. H. (2017) Evaluating Efficiencies of Dual AAV Approaches for Retinal Targeting. Front Neurosci, 11, 503.
20 Dyka, F. M., Molday, L. L., Chiodo, V. A., Molday, R. S. and Hauswirth, W. W. (2019) Dual ABCA4-AAV Vector Treatment Reduces Pathogenic Retinal A2E Accumulation in a Mouse Model of autosomal Recessive Stargardt Disease. Hum Gene Ther, 30, 1361-1370.
21 Barrett, B., Kerns, S., Kevany, B. and Padegimas, L. (2021) Dual AAV vector strategy for expression of large genes targeted for Stargardt disease gene therapy development. Invest Ophthalmol Vis Sci, 62, 1484.
22 Riedmayr, L. M., Hinrichsmeyer, K. S., Thalhammer, S. B., Mittas, D. M., Karguth, N., Otify, D. Y., Bohm, S., Weber, V. J., Bartoschek, M. D., Splith, V. et al. (2023) mRNA trans-splicing dual AAV vectors for (epi)genome editing and gene therapy. Nat Commun, 14, 6578.
23 Shah, N. H. and Muir, T. W. (2014) Inteins: Nature's Gift to Protein Chemists. Chem Sci, 5, 446-461.
24 Wu, H., Hu, Z. and Liu, X. Q. (1998) Protein trans-splicing by a split intein encoded in a split DnaE gene of Synechocystis sp. PCC6803. Proc Natl Acad Sci USA, 95, 9226-9231.
25 Iwai, H., Zuger, S., Jin, J. and Tam, P. H. (2006) Highly efficient protein trans-splicing by a naturally split DnaE intein from Nostoc punctiforme. FEBS Lett, 580, 1853-1858.
26 Trapani, I., Colella, P., Sommella, A., Iodice, C., Cesi, G., de Simone, S., Marrocco, E., Rossi, S., Giunti, M., Palfi, A. et al. (2014) Effective delivery of large genes to the retina by dual AAV vectors. EMBO Mol Med, 6, 194-211.
27 Trapani, I., Toriello, E., de Simone, S., Colella, P., Iodice, C., Polishchuk, E. V., Sommella, A., Colecchi, L., Rossi, S., Simonelli, F. et al. (2015) Improved dual AAV vectors with reduced expression of truncated proteins are safe and effective in the retina of a mouse model of Stargardt disease. Hum Mol Genet, 24, 6811-6825
28 McClements, M. E., Barnard, A. R., Singh, M. S., Charbel Issa, P., Jiang, Z., Radu, R. A. and MacLaren, R. E. (2019) An AAV Dual Vector Strategy Ameliorates the Stargardt Phenotype in Adult Abca4(−/−) Mice. Hum Gene Ther, 30, 590-600.
29 Villiger, L., Grisch-Chan, H. M., Lindsay, H., Ringnalda, F., Pogliano, C. B., Allegri, G., Fingerhut, R., Haberle, J., Matos, J., Robinson, M. D. et al. (2018) Treatment of a metabolic liver disease by in vivo genome base editing in adult mice. Nat Med, 24, 1519-1525.
30 Tornabene, P., Trapani, I., Minopoli, R., Centrulo, M., Lupo, M., de Simone, S., Tiberi, P., Dell'Aquila, F., Marrocco, E., Iodice, C. et al. (2019) Intein-mediated protein trans-splicing expands adenoassociated virus transfer capacity in the retina. Sci Transl Med, 11.
31 Chong, S., Williams, K. S., Wotkowicz, C. and Xu, M. Q. (1998) Modulation of protein splicing of the Saccharomyces cerevisiae vacuolar membrane ATPase intein. J Biol Chem, 273, 10567-10577.
32 Shah, N. H., Eryilmaz, E., Cowburn, D. and Muir, T. W. (2013) Extein residues play an intimate role in the rate-limiting step of protein trans-splicing. J Am Chem Soc, 135, 5839-5847.
33 Hoess, R. H. and Abremski, K. (1984) Interaction of the bacteriophage P1 recombinase Cre with the recombining site loxP. Proc Natl Acad Sci USA, 81, 1026-1029.
34 Hoess, R. H., Wierzbicki, A. and Abremski, K. (1986) The role of the loxP spacer region in P1 sitespecific recombination. Nucleic Acids Res, 14, 2287-2300.
35 Hoess, R. H. and Abremski, K. (1985) Mechanism of strand cleavage and exchange in the Cre-lox site-specific recombination system. J Mol Biol, 181, 351-362.
36 Lee, G. and Saito, I. (1998) Role of nucleotide sequences of loxP spacer region in Cre-mediated recombination. Gene, 216, 55-65.
37 Siegel, R. W., Jain, R. and Bradbury, A. (2001) Using an in vivo phagemid system to identify noncompatible loxP sequences. FEBS Lett, 505, 467-473
38 Livet, J., Weissman, T. A., Kang, H., Draft, R. W., Lu, J., Bennis, R. A., Sanes, J. R. and Lichtman, J. W. (2007) Transgenic strategies for combinatorial expression of fluorescent proteins in the nervous system. Nature, 450, 56-62.
39 Missirlis, P. I., Smailus, D. E. and Holt, R. A. (2006) A high-throughput screen identifying sequence and promiscuity characteristics of the loxP spacer region in Cre-mediated recombination. BMC Genomics, 7, 73.
40 Thomson, J. G., Rucker, E. B., 3rd and Piedrahita, J. A. (2003) Mutational analysis of loxP sites for efficient Cre-mediated insertion into genomic DNA. Genesis, 36, 162-167.
41 Albert, H., Dale, E. C., Lee, E. and Ow, D. W. (1995) Site-specific integration of DNA into wildtype and mutant lox sites placed in the plant genome. Plant J, 7, 649-659.
42 Xu, M., Yang, L., Wang, F., Li, H., Wang, X., Wang, W., Ge, Z., Wang, K., Zhao, L., Li, H. et al. (2015) Mutations in human IFT140 cause non-syndromic retinal degeneration. Hum Genet, 134, 1069-1078. virus transfer capacity in the retina. Sci Transl Med, 11.
43 Hull, S., Owen, N., Islam, F., Tracey-White, D., Plagnol, V., Holder, G. E., Michaelides, M., Carss, K., Raymond, F. L., Rozet, J. M. et al. (2016) Nonsyndromic Retinal Dystrophy due to Bi-Allelic Mutations in the Ciliary Transport Gene IFT140. Invest Ophthalmol Vis Sci, 57, 1053-1062.
44 Perrault, I., Saunier, S., Hanein, S., Filhol, E., Bizet, A. A., Collins, F., Salih, M. A., Gerber, S., Delphin, N., Bigot, K. et al. (2012) Mainzer-Saldino syndrome is a ciliopathy caused by IFT140 mutations. Am J Hum Genet, 90, 864-870.
45 Earley, L. F., Conatser, L. M., Lue, V. M., Dobbins, A. L., Li, C., Hirsch, M. L. and Samulski, R. J. (2020) Adeno-Associated Virus Serotype-Specific Inverted Terminal Repeat Sequence Role in Vector Transgene Expression. Hum Gene Ther, 31, 151-162.
46 Khani, S. C., Pawlyk, B. S., Bulgakov, O. V., Kasperek, E., Young, J. E., Adamian, M., Sun, X., Smith, A. J., Ali, R. R. and Li, T. (2007) AAV-mediated expression targeting of rod and cone photoreceptors with a human rhodopsin kinase promoter. Invest Ophthalmol Vis Sci, 48, 3954-3961.
47 Jonassen, J. A., SanAgustin, J., Baker, S. P. and Pazour, G. J. (2012) Disruption of IFT complex A causes cystic kidneys without mitotic spindle misorientation. J Am Soc Nephrol, 23, 641-651.
48 Crouse, J. A., Lopes, V. S., Sanagustin, J. T., Keady, B. T., Williams, D. S. and Pazour, G. J. (2014) Distinct functions for IFT140 and IFT20 in opsin transport. Cytoskeleton (Hoboken), 71, 302-310.
49 Li, S., Chen, D., Sauve, Y., McCandless, J., Chen, Y. J. and Chen, C. K. (2005) Rhodopsin-iCre transgenic mouse line for Cre-mediated rod-specific gene targeting. Genesis, 41, 73-80.
50 Ahmed, Z. M., Riazuddin, S., Bernstein, S. L., Ahmed, Z., Khan, S., Griffith, A. J., Morell, R. J., Friedman, T. B., Riazuddin, S. and Wilcox, E. R. (2001) Mutations of the protocadherin gene PCDH15 cause Usher syndrome type 1F. Am J Hum Genet, 69, 25-34.
51 Alagramam, K. N., Murcia, C. L., Kwon, H. Y., Pawlowski, K. S., Wright, C. G. and Woychik, R. P. (2001) The mouse Ames waltzer hearing-loss mutant is caused by mutation of Pcdh15, a novel protocadherin gene. Nat Genet, 27, 99-102.
52 Alagramam, K. N., Yuan, H., Kuehn, M. H., Murcia, C. L., Wayne, S., Srisailpathy, C. R., Lowry, R. B., Knaus, R., Van Laer, L., Bernier, F. P. et al. (2001) Mutations in the novel protocadherin PCDH15 cause Usher syndrome type 1F. Hum Mol Genet, 10, 1709-1718.
53 Chang, B., Khanna, H., Hawes, N., Jimeno, D., He, S., Lillo, C., Parapuram, S. K., Cheng, H., Scott, A., Hurd, R. E. et al. (2006) In-frame deletion in a novel centrosomal/ciliary protein CEP290/NPHP6 perturbs its interaction with RPGR and results in early-onset retinal degeneration in the rd16 mouse. Hum Mol Genet, 15, 1847-1857.
54 Cideciyan, A. V., Aleman, T. S., Jacobson, S. G., Khanna, H., Sumaroka, A., Aguirre, G. K., Schwartz, S. B., Windsor, E. A., He, S., Chang, B. et al. (2007) Centrosomal-ciliary gene CEP290/NPHP6 mutations result in blindness with unexpected sparing of photoreceptors and visual brain: implications for therapy of Leber congenital amaurosis. Human mutation, 28, 1074-1083.
55 Frank, V., den Hollander, A. I., Bruchle, N. O., Zonneveld, M. N., Nurnberg, G., Becker, C., Du Bois, G., Kendziorra, H., Roosing, S., Senderek, J. et al. (2008) Mutations of the CEP290 gene encoding a centrosomal protein cause Meckel-Gruber syndrome. Hum Mutat, 29, 45-52
56 Iwamoto, M., Bjorklund, T., Lundberg, C., Kirik, D. and Wandless, T. J. (2010) A general chemical method to regulate protein stability in the mammalian central nervous system. Chem Biol, 17, 981-988.
57 Sando, R., 3rd, Baumgaertel, K., Pieraut, S., Torabi-Rander, N., Wandless, T. J., Mayford, M. and Maximov, A. (2013) Inducible control of gene expression with destabilized Cre. Nat Methods, 10, 1085-1088.
58 Schmidt, E. E., Taylor, D. S., Prigge, J. R., Barnett, S. and Capecchi, M. R. (2000) Illegitimate Cre dependent chromosome rearrangements in transgenic mouse spermatids. Proc Natl Acad Sci USA, 97, 13702-13707.
59 Seo, S., Guo, D. F., Bugge, K., Morgan, D. A., Rahmouni, K. and Sheffield, V. C. (2009) Requirement of Bardet-Biedl Syndrome Proteins for Leptin Receptor Signaling. Hum Mol Genet, 18, 1323-1331.
60 Seo, S., Zhang, Q., Bugge, K., Breslow, D. K., Searby, C. C., Nachury, M. V. and Sheffield, V. C. (2011) A Novel Protein LZTFL1 Regulates Ciliary Trafficking of the BBSome and Smoothened. PLOS genetics, 7, e1002358.
61 Datta, P., Hendrickson, B., Brendalen, S., Ruffcorn, A. and Seo, S. (2019) The myosin-tail homology domain of centrosomal protein 290 is essential for protein confinement between the inner and outer segments in photoreceptors. J Biol Chem, 294, 19119-19136.
62 Humbert, M. C., Weihbrecht, K., Searby, C. C., Li, Y., Pope, R. M., Sheffield, V. C. and Seo, S. (2012) ARL13B, PDE6D, and CEP164 form a functional network for INPPSE ciliary targeting. Proceedings of the National Academy of Sciences of the United States of America, 109, 19691-19696.
63 Datta, P., Ruffcorn, A. and Seo, S. (2020) Limited time window for retinal gene therapy in a preclinical model of ciliopathy. Hum Mol Genet, 29, 2337-2352.
64 Datta, P., Cribbs, J. T. and Seo, S. (2021) Differential requirement of NPHP1 for compartmentalized protein localization during photoreceptor outer segment development and maintenance. PLOS One, 16, e0246358.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. An adeno-associated virus (AAV) vector set for delivering a gene to a target cell comprising:
at least two recombinant AAV vectors, each vector comprising an AAV inverted terminal repeat (ITR) at each end of a vector genome and a respective fragment of a coding sequence of the gene; and
matched hybrid lox site pairs, each pair comprising two hybrid lox sites respectively flanking adjacent fragments of the coding sequence in the at least two vectors, each hybrid lox site comprising:
an inverted repeat element having a mutation in only one of its left or right elements; and
a non-compatible spacer sequence that prevents recombination with any other hybrid lox site other than its matched partner.

2. The AAV vector set of claim 1, further comprising a promoter operably linked to the fragment in a first AAV vector of the at least two vectors.

3. The AAV vector set of claim 1, further comprising a transcription termination signal operably linked downstream of the fragment in a last AAV vector of the at least two vectors.

4. The AAV vector set of claim 2, wherein the promoter comprises a human cytomegalovirus immediate-early (CMV) promoter or a a rod photoreceptor-specific rhodopsin kinase (GRK1) promoter.

5. The AAV vector set of claim 2, wherein the transcription termination signal comprises a polyadenylation signal.

6. The AAV vector set of claim 1, wherein the inverted repeat element of each hybrid lox site comprises a mutation in the left element only.

7. The AAV vector set of claim 1, wherein the non-compatible spacer sequence comprises the nucleotide sequence of lox2272.

8. The AAV vector set of claim 1, wherein each matched hybrid lox site pair comprises a loxJT15 site and a loxJTZ17 site.

9. The AAV vector set of claim 1, wherein the at least two vectors consist of two, three or four vectors.

10. The AAV vector set of claim 1, wherein one of the at least two vectors further comprises a coding sequence encoding CRE recombinase.

11. The AAV vector set of claim 10, wherein CRE recombinase coding sequence is downstream of the matched hybrid lox site.

12. The AAV vector set of claim 10, further comprising a self-cleaving peptide.

13. The AAV vector set of claim 12, wherein the a self-cleaving peptide is a T2A self-cleaving peptide.

14. The AAV vector set of claim 1, wherein the gene encodes the human intraflagellar transport protein 140 (IFT140), PCDH15, CEP290 or CDH23.

15. A method to reconstitute a fragmented coding sequence in a target cell comprising:
co-administering at least two recombinant adeno-associated virus (AAV) vectors to the cell, each vector comprising AAV inverted terminal repeats (ITRs) flanking a distinct fragment of the coding sequence and a hybrid lox site at one end of said fragment, wherein adjacent fragments are flanked by matched hybrid lox site pairs, each hybrid lox site comprising an inverted repeat element mutated in only one of its left or right elements and a non-compatible spacer sequence matched only to its corresponding hybrid lox site;
expressing CRE recombinase in the target cell; and
permitting CRE recombinase to mediate unidirectional, sequence-specific recombination between each matched hybrid lox site pair to reconstitute the distinct fragments into a contiguous, full-length coding sequence of the gene in a predetermined order.

16. The method of claim 15, wherein the at least two vectors consist of two, three or four vectors.

17. The method of claim 15, wherein one of the at least two vectors further comprises a coding sequence encoding CRE recombinase downstream of the matched hybrid lox site and a T2A self-cleaving peptide operably linking the coding sequence.

18. The method of claim 15, further comprising a promoter, wherein the promoter comprises a human cytomegalovirus immediate-early (CMV) promoter or a rod photoreceptor-specific rhodopsin kinase (GRK1) promoter.

19. The method of claim 15, further comprising a transcription termination signal, wherein the transcription termination signal optionally comprises a polyadenylation signal.

20. A method to treat or prevent a disease, disorder or cancer, comprising administering the AAV vector set of claim 1 to a subject in need thereof.

21. The method of claim 20, wherein the disorder is retinal regeneration, and the vector set comprises a fragmented IFT140 gene resulting in expression of a full length IFT140 protein in said subject.


SEQUENCE LISTING (29 sequences)

SEQ ID NO: 1 - DNA, 34 bp, synthetic construct
  source 1..34: mol_type = other DNA
ataacttcgtatagcatacattatacgaagttat

SEQ ID NO: 2 - DNA, 34 bp, synthetic construct
  source 1..34: mol_type = other DNA
ataacttcgtatagtataccttatacgaagttat

SEQ ID NO: 3 - DNA, 34 bp, synthetic construct
  source 1..34: mol_type = other DNA
ataacttcgtataggatactttatacgaagttat

SEQ ID NO: 4 - DNA, 34 bp, synthetic construct
  source 1..34: mol_type = other DNA
ataacttcgtatattctatcttatacgaagttat

SEQ ID NO: 5 - DNA, 34 bp, synthetic construct
  source 1..34: mol_type = other DNA
ataacttcgtatactatagcctatacgaagttat

SEQ ID NO: 6 - DNA, 34 bp, synthetic construct
  source 1..34: mol_type = other DNA
ataacttcgtatatactatactatacgaagttat

SEQ ID NO: 7 - DNA, 34 bp, synthetic construct; identical to SEQ ID NO: 1
  source 1..34: mol_type = other DNA

SEQ ID NO: 8 - DNA, 34 bp, synthetic construct
  source 1..34: mol_type = other DNA
aattattcgtatagcatacattatacgaagttat

SEQ ID NO: 9 - DNA, 34 bp, synthetic construct
  source 1..34: mol_type = other DNA
ataacttcgtatagcatacattatagcaatttat

SEQ ID NO: 10 - DNA, 34 bp, synthetic construct
  source 1..34: mol_type = other DNA
aattattcgtatagtataccttatacgaagttat

SEQ ID NO: 11 - protein, 34 aa, synthetic construct
ATAACTTCGTATAGTATACCTTATAGCAATTTAT

SEQ ID NO: 12 - DNA, 34 bp, synthetic construct
  source 1..34: mol_type = other DNA
aattattcgtatagtataccttatagcaatttat

SEQ ID NO: 13 - DNA, 34 bp, synthetic construct; identical to SEQ ID NO: 2
  source 1..34: mol_type = other DNA

SEQ ID NO: 14 - DNA, 34 bp, synthetic construct
  source 1..34: mol_type = other DNA
aattattcgtataggatactttatacgaagttat

SEQ ID NO: 15 - DNA, 34 bp, synthetic construct
  source 1..34: mol_type = other DNA
ataacttcgtataggatactttatagcaatttat

SEQ ID NO: 16 - DNA, 34 bp, synthetic construct
  source 1..34: mol_type = other DNA
aattattcgtataggatactttatagcaatttat

SEQ ID NO: 17 - DNA, 34 bp, synthetic construct; identical to SEQ ID NO: 3
  source 1..34: mol_type = other DNA

SEQ ID NO: 18 - DNA, 34 bp, synthetic construct
  source 1..34: mol_type = other DNA
aattattcgtatactatagcctatacgaagttat

SEQ ID NO: 19 - DNA, 34 bp, synthetic construct
  source 1..34: mol_type = other DNA
ataacttcgtatactatagcctatagcaatttat

SEQ ID NO: 20 - DNA, 34 bp, synthetic construct
  source 1..34: mol_type = other DNA
aattattcgtatactatagcctatagcaatttat

SEQ ID NO: 21 - DNA, 34 bp, synthetic construct; identical to SEQ ID NO: 5
  source 1..34: mol_type = other DNA

SEQ ID NO: 22 - DNA, 34 bp, synthetic construct
  source 1..34: mol_type = other DNA
aattattcgtatatactatactatacgaagttat

SEQ ID NO: 23 - DNA, 34 bp, synthetic construct
  source 1..34: mol_type = other DNA
ataacttcgtatatactatactatagcaatttat

SEQ ID NO: 24 - DNA, 34 bp, synthetic construct
  source 1..34: mol_type = other DNA
aattattcgtatatactatactatagcaatttat

SEQ ID NO: 25 - DNA, 34 bp, synthetic construct; identical to SEQ ID NO: 6
  source 1..34: mol_type = other DNA

SEQ ID NO: 26 - DNA, 34 bp, synthetic construct
  source 1..34: mol_type = other DNA
ataacttcgtatagtataccttatagcaatttat

SEQ ID NO: 27 - DNA, 34 bp, synthetic construct; identical to SEQ ID NO: 15
  source 1..34: mol_type = other DNA

SEQ ID NO: 28 - DNA, 34 bp, synthetic construct; identical to SEQ ID NO: 8
  source 1..34: mol_type = other DNA

SEQ ID NO: 29 - DNA, 34 bp, synthetic construct; identical to SEQ ID NO: 14
  source 1..34: mol_type = other DNA